(19) United States
(12) Patent Application Publication
(10) Pub. No.: US 2018/0149617 A1
(43) Pub. Date: May 31, 2018

(54) GAS DETECTOR AND CONTROL METHOD FOR GAS DETECTOR

(71) Applicant: Toyota Jidosha Kabushiki Kaisha, Toyota-shi Aichi-ken (JP)

(72) Inventors: Kazuhiro Wakao, Susono-shi Shizuoka-ken (JP); Keiichiro Aoki, Sunto-gun Shizuoka-ken (JP); Kazuhisa Matsuda, Susono-shi Shizuoka-ken (JP)

(21) Appl. No.: 15/802,988

(22) Filed: Nov. 3, 2017

(30) Foreign Application Priority Data
Nov. 30, 2016 (JP) 2016-233384

(51) Int. Cl. G01N 27/407; G01M 15/10; G01N 27/406; G01N 27/419; G01N 33/00

(52) U.S. Cl. CPC G01N 27/4074; G01M 15/102; G01N 27/4067; G01N 27/41; G01N 27/4065; G01N 33/0042; G01N 27/419

(57) ABSTRACT

A gas detector includes an electronic control unit. The electronic control unit obtains a value correlated with an output current flowing between a first electrode and a second electrode in a period in which lowering sweep is executed and in which an applied voltage is equal to or lower than a decomposition initiation voltage of sulfur oxides as a first current. The electronic control unit obtains the output current that is detected when the applied voltage is a particular voltage that is equal to or higher than a voltage at which the output current is a limiting current of oxygen, and that exclude a current resulted from a reoxidation reaction of sulfur in the first electrode by boosting sweep as a second current. The electronic control unit detects a concentration of sulfur oxides based on a difference between the second current and the first current.

### CROSS-REFERENCE TO RELATED APPLICATION

This application claims priority to Japanese Patent Application No. 2016-233384 filed on Nov. 30, 2016, the entire contents of which are incorporated herein by reference.

### BACKGROUND

### 1. Technical Field

The disclosure relates to a gas detector capable of determining presence or absence of sulfur oxides in a predetermined concentration or higher that are contained in exhaust gas discharged from an internal combustion engine or detecting a concentration of sulfur oxides contained in the exhaust gas, and to a control method for a gas detector.

### 2. Description of Related Art

In order to control an internal combustion engine, an air-fuel ratio sensor (hereinafter also referred to as an “A/F sensor”) that obtains an air-fuel ratio (A/F) of air mixture in a combustion chamber on the basis of a concentration of oxygen (O₂) contained in exhaust gas has widely been used. One type of such an air-fuel ratio sensor is a limiting current type gas sensor.

A SOx concentration detector that detects a concentration of sulfur oxides (hereinafter may also be referred to as “SOx”) in the exhaust gas by using the limiting current type gas sensor has been proposed in Japanese Patent Application Publication No. 2015-17931 (JP 2015-17931 A).

The SOx concentration detector in JP 2015-17931 A includes a sensing cell (an electrochemical cell) that uses an oxygen pumping effect of an oxygen ion conductive solid electrolyte. This SOx concentration detector applies a voltage between paired electrodes of the sensing cell so as to decompose gas components, which includes oxygen atoms, in the exhaust gas, and thereby generates oxide ions (O²⁻). The gas components, which include oxygen atoms, in the exhaust gas are O₂, SOx, H₂O, and the like, for example, and hereinafter will also be referred to as “oxygen containing components”. The SOx concentration detector detects a characteristic of a current flowing between the electrodes of the sensing cell when the oxide ions, which are generated through decomposition of the oxygen containing components, move between the electrodes (the oxygen pumping effect).

More specifically, in JP 2015-17931 A, the SOx concentration detector executes applied voltage sweep when detecting the SOx concentration. That is, this SOx concentration detector executes the applied voltage sweep in which the applied voltage to the sensing cell is boosted from 0.4 V to 0.8 V and is thereafter lowered from 0.8 V to 0.4 V.

Then, in JP 2015-17931 A, the SOx concentration is calculated by using a difference between a reference current as a “current flowing between the electrodes of the sensing cell (hereinafter may also be referred to as an “electrode current” or an “output current”)” at a time point at which the applied voltage reaches 0.8 V and a peak value as a minimum value of the output current in a period in which the applied voltage is lowered from 0.8 V to 0.4 V.

### SUMMARY

However, there is a case where the above output current is changed under an influence of the oxygen containing component other than SOx contained in the exhaust gas. For example, a decomposition voltage of water (H₂O) is approximately the same as or slightly higher than a decomposition voltage of sulfur oxides. Furthermore, a concentration of water in the exhaust gas fluctuates in accordance with the air-fuel ratio of the air mixture, for example. For this reason, it is difficult to eliminate the influence to the output current resulted from the decomposition of water and to detect the output current only resulted from the decomposition of SOx components. Accordingly, it has been desired to determine whether sulfur oxides in a predetermined concentration or higher exist in the exhaust gas or to detect the concentration of sulfur oxides in the exhaust gas by using a change in the output current that is not influenced by the oxygen containing components other than SOx and that is only caused by the SOx components.

The disclosure provides a gas detector and a control method for a gas detector that are capable of accurately determining whether sulfur oxides in a predetermined concentration or higher are contained in exhaust gas or detecting a concentration of sulfur oxides in the exhaust gas.

A first aspect of the disclosure provides a gas detector. The gas detector includes an element, a voltage application device, a current detector, and an electronic control unit. The element is provided in an exhaust passage of an internal combustion engine. The element includes an electrochemical cell and a diffusion resistance body. The electrochemical cell includes a solid electrolyte body, a first electrode, and a second electrode. The solid electrolyte body has oxide ion conductivity. The first electrode and the second electrode are respectively provided on surfaces of the solid electrolyte body. The diffusion resistance body is made of a porous material through which exhaust gas flowing through the exhaust passage can pass. The element is configured that the exhaust gas flowing through the exhaust passage reaches the first electrode through the diffusion resistance body. The voltage application device is configured to apply a voltage between the first electrode and the second electrode. The current detector is configured to detect an output current that is a current flowing between the first electrode and the second electrode. The electronic control unit is configured to control an applied voltage that is the voltage applied between the first electrode and the second electrode by the voltage application device. The electronic control unit is configured to either determine whether sulfur oxides in a predetermined concentration or higher are contained in the exhaust gas or detect a concentration of sulfur oxides in the exhaust gas, based on the output current detected by the current detector. When an air-fuel ratio of air mixture supplied to the internal combustion engine is in a stable state, the electronic control unit is configured to execute boosting sweep for boosting the applied voltage from a predetermined voltage to a second voltage by the voltage application device and then execute lowering sweep for lowering the applied voltage from the second voltage to a first voltage at a predetermined lowering rate. The predetermined voltage is a voltage that is equal to or higher than the first voltage and is lower than a decomposition initiation voltage of sulfur oxides. The first voltage is a voltage that is lower than the decomposition initiation voltage of sulfur oxides. The first voltage is a voltage when the output current becomes a limiting current of oxygen. The second voltage is a voltage that is higher than the decomposition initiation voltage of sulfur oxides. The electronic control unit is configured to obtain a parameter that is correlated with a degree of a change occurred to the output current based on the output current detected by the current detector. The change occurred to the output current is a change in the output current resulted from the current flowing between the first electrode and the second electrode when a reoxidation reaction of sulfur that has been adsorbed to the first electrode leads to generation of sulfur oxides in the first electrode when the applied voltage becomes lower than the decomposition initiation voltage of sulfur oxides during the lowering sweep. The degree of the change occurred to the output current is increased as the concentration of sulfur oxides contained in the exhaust gas is increased. The electronic control unit is configured to either determine whether sulfur oxides in the predetermined concentration or higher are contained in the exhaust gas or detect the concentration of sulfur oxides in the exhaust gas, based on the parameter. The electronic control unit is configured to set the predetermined lowering rate such that a rate of the reoxidation reaction becomes a rapidly increased rate when the applied voltage becomes a voltage within a range between the first voltage and the decomposition initiation voltage of sulfur dioxides. The electronic control unit is configured to obtain a value that is correlated with the output current in a predetermined period as a first current based on the output current detected by the current detector. The predetermined period is a period in which the lowering sweep is executed and in which the applied voltage is within a range between the first voltage and the decomposition initiation voltage of sulfur oxides. The first voltage is excluded from the range. The decomposition initiation voltage is included in the range. The electronic control unit is configured to obtain a second current. The second current is the output current detected by the current detector at a time when the applied voltage is a particular voltage that is equal to or higher than a voltage at which the output current is the limiting current of oxygen, and the second current is the output current that exclude a current resulted from the reoxidation reaction of sulfur that has been adsorbed to the first electrode in the first electrode by the boosting sweep. The electronic control unit is configured to calculate a difference between the obtained second current and the obtained first current and use the difference as the parameter.

According to the investigation by the inventor, it has been found that the change in the output current, which is less likely to be influenced by oxygen containing components other than sulfur oxides, occurs due to the reoxidation reaction of sulfur, which has been adsorbed to the first electrode, in the first electrode leads to generation of sulfur oxides during the lowering sweep. Furthermore, it has been found that the degree of this change in the output current is significantly changed by a voltage lowering amount (that is, a lowering rate) per predetermined elapsed time during the lowering sweep (see FIG. 5A and FIG. 5B). A mechanism of causing such phenomena is estimated as follows.

More specifically, during the lowering sweep, the reoxidation reaction of sulfur (decomposed matters of sulfur oxides), which has been adsorbed to the first electrode by the boosting sweep, leads to the generation of sulfur oxides in the first electrode. When the boosting sweep is executed, the decomposed matters of the oxygen containing components other than sulfur oxides (for example, hydrogen as the decomposed matter of water) are not adsorbed to the first electrode. Accordingly, such a phenomenon that the reoxidation reaction of the decomposed matters of the oxygen containing components other than sulfur oxides leads to generation of the oxygen containing components in the first electrode during the lowering sweep does not substantially occur.

Thus, the change in the output current, which is resulted from the reoxidation reaction of sulfur adsorbed to the first electrode leads to the generation of sulfur oxides in the first electrode during the lowering sweep, is less likely to be influenced by the oxygen containing components other than sulfur oxides. That is, the change in the output current that is less likely to be influenced by oxygen containing components during the lowering sweep occurs.

However, when the lowering rate (a sweeping rate) of the lowering sweep is lower than a certain rate, the reoxidation reaction of sulfur is continuously and gradually progressed during the lowering sweep. Accordingly, regardless of the concentration of sulfur oxides, the change in the output current is less likely to appear.

On the other hand, when the lowering rate of the lowering sweep is increased to be higher than the certain rate, the applied voltage is lowered while the reoxidation reaction of sulfur is not significantly progressed during the lowering sweep. Then, when the applied voltage becomes the voltage that is within the voltage range where the reoxidation reaction of sulfur becomes active (that is, a voltage range that is less than the decomposition initiation voltage of sulfur oxides), the reoxidation reaction of sulfur is rapidly progressed (a rate of the reoxidation reaction of sulfur is rapidly increased, and a frequency of the reoxidation reaction of sulfur is rapidly increased). Accordingly, as the concentration of sulfur oxides is increased, the degree of the change in the output current is increased. That is, the current change, which yields a significant effect on accurate detection of the concentration of sulfur oxides, appears.

For the above reason, in the above gas detector, the lowering rate in the lowering sweep is set such that the rate of the reoxidation reaction of sulfur becomes a rapidly increased rate at a time point at which the applied voltage becomes a voltage within a voltage range between the first voltage and the decomposition initiation voltage of sulfur oxides. Accordingly, the change in the output current that is not influenced by the oxygen containing components other than sulfur oxides appears significantly as the concentration of sulfur oxides is increased.

Furthermore, the above gas detector is configured to obtain the parameter, which is correlated with the degree of the change occurred to the output current resulted from such a reoxidation reaction of sulfur, based on the output current and to either determine whether sulfur oxides in the predetermined concentration or higher are contained in the exhaust gas or detect the concentration of sulfur oxides in the exhaust gas on the basis of the parameter.

Moreover, the above gas detector adopts the difference between the second current and the first current as the parameter that represents the reoxidation current change. The first current has such characteristics that the first current is changed in accordance with the oxygen concentration in the exhaust gas and that the first current is decreased as the concentration of sulfur oxides in the exhaust gas is increased. The second current has such characteristics that the second current is changed in accordance with the oxygen concentration in the exhaust gas and that the second current is not changed in accordance with the concentration of sulfur oxides in the exhaust gas. That is, the magnitude of the second current remains the same regardless of the concentration of sulfur oxides in the exhaust gas.

The magnitude of the second current remains the same regardless of the concentration of sulfur oxides in the exhaust gas. Meanwhile, as the concentration of sulfur oxides in the exhaust gas is increased, a degree of the reoxidation current change becomes significant, and the first current is decreased. Accordingly, as the concentration of sulfur oxides in the exhaust gas is increased, a magnitude of the difference is also increased. In addition, the first current is changed under an influence of the oxygen concentration in the exhaust gas, and a degree of the influence also appears in a similar manner to the second current. Accordingly, the difference is not influenced by the oxygen concentration (the engine air-fuel ratio) in the exhaust gas and is the parameter that accurately represents the concentration of sulfur oxides.

The detector of the disclosure uses this parameter (the above difference) to either determine whether sulfur oxides in the predetermined concentration or higher are contained in the exhaust gas or detect the concentration of sulfur oxides in the exhaust gas. Thus, such a determination or detection of the concentration can accurately be executed.

In the gas detector, the electronic control unit may be configured to determine whether sulfur oxides in the predetermined concentration or higher are contained in the exhaust gas. The electronic control unit may determine whether a magnitude of the difference is equal to or larger than a predetermined threshold. The electronic control unit may be configured to determine that sulfur oxides in the predetermined concentration or higher are contained in the exhaust gas when the electronic control unit determines that the magnitude of the difference is equal to or larger than the predetermined threshold. The electronic control unit may be configured to determine that sulfur oxides in the predetermined concentration or higher are not contained in the exhaust gas when the electronic control unit determines that the magnitude of the difference is lower than the predetermined threshold.

With this configuration, it is determined whether the above magnitude of the difference, which accurately represents the concentration of sulfur oxides, is equal to or larger than the “threshold corresponding to the predetermined concentration”. Therefore, it is possible to accurately determine whether sulfur oxides in the predetermined concentration or higher are contained in the exhaust gas.

In the gas detector, the electronic control unit may be configured to detect the concentration of sulfur oxides in the exhaust gas. The electronic control unit may be configured to detect the concentration of sulfur oxides in the exhaust gas based on the difference.

In the above case, the concentration of sulfur oxides in the exhaust gas is detected on the basis of the difference, which accurately represents the concentration of sulfur oxides. Therefore, the concentration of sulfur oxides in the exhaust gas can easily be detected.

In the gas detector, the electronic control unit may be configured to obtain a minimum value of the output current detected by the current detector in a period in which the lowering sweep is executed and in which the applied voltage is in a detection voltage range as the first current. The detection voltage range may be a range between a fourth voltage and a third voltage inclusive. The third voltage may be a voltage that is equal to or lower than the decomposition initiation voltage of the sulfur oxides. The fourth voltage may be a voltage that is higher than the first voltage.

The minimum value of the output current in the period in which the applied voltage is the voltage within the above detection voltage range (that is, a period in which the reoxidation reaction of sulfur is active) accurately represents the concentration of sulfur oxides. This minimum value is used as the first current, and thereby the above difference is the value that accurately represents the concentration of sulfur oxides. Accordingly, it is possible to accurately determine whether sulfur oxides in the predetermined concentration or higher are contained in exhaust gas or detect the concentration of sulfur oxides in the exhaust gas.

In the gas detector, the electronic control unit may be configured to obtain the output current detected by the current detector when the lowering sweep is executed and the applied voltage becomes a current obtainment voltage as the first current. The current obtainment voltage may be a voltage selected from a detection voltage range. The detection voltage range may be a range between a fourth voltage and a third voltage inclusive. The third voltage may be a voltage that is equal to or lower than the decomposition initiation voltage of sulfur oxides. The fourth voltage may be a voltage that is higher than the first voltage.

The output current when the applied voltage becomes the current obtainment voltage selected from the above detection voltage range accurately represents the concentration of sulfur oxides. This output current is used as the above first current, and thereby the above difference accurately represents the concentration of sulfur oxides. Accordingly, it is possible to accurately determine whether sulfur oxides in the predetermined concentration or higher are contained in exhaust gas or detect the concentration of sulfur oxides in the exhaust gas.

In the gas detector, the electronic control unit may be configured to adopt an applied voltage for detection of an air-fuel ratio, at which the output current becomes the limiting current of oxygen, as the particular voltage. The electronic control unit may be configured to set the applied voltage to the applied voltage for the detection of the air-fuel ratio by the voltage application device before starting execution of the boosting sweep. The electronic control unit may be configured to obtain the output current that is detected by the current detector when the applied voltage is set as the applied voltage for the detection of the air-fuel ratio as the second current.

According to this aspect, when the applied voltage is set as the applied voltage for the detection of the air-fuel ratio and the output current is the limiting current of oxygen, the output current is obtained as the second current. An amount of the current that corresponds to the limiting current of oxygen is included in the first current. Accordingly, the difference between the second current and the first current, which is obtained as described above, becomes the parameter that is less likely to be influenced by the oxygen concentration in the exhaust gas. Thus, the difference becomes the parameter that accurately represents the concentration of the sulfur oxides. As a result of this, the gas detector of this aspect can accurately determine whether sulfur oxides in the predetermined concentration or higher are contained in exhaust gas or detect the concentration of sulfur oxides in the exhaust gas.

In the gas detector, the electronic control unit may be configured to obtain the output current detected by the current detector when the boosting sweep is executed and the applied voltage becomes the second voltage as the second current.

In the gas detector, the electronic control unit may be configured to obtain the output current detected by the current detector when the lowering sweep is executed and the applied voltage becomes the first voltage as the second current.

In each of these cases, the second current can be obtained at the time point when the lowering sweep is initiated or terminated, and the first current can be obtained during the same lowering sweep. In this way, both of the “first current and the second current”, which are required to obtain the parameter, can be obtained in a short period.

Accordingly, there is a low possibility that the oxygen concentration in the exhaust gas is significantly changed in the period. Thus, degrees of the influence of the oxygen concentration in the exhaust gas on the first current and the second current can substantially match each other. As a result, the difference becomes a value that is less likely to be influenced by the oxygen concentration in the exhaust gas and that accurately corresponds to the concentration of sulfur oxides in the exhaust gas. Therefore, it is possible to further accurately determine whether sulfur oxides in the predetermined concentration or higher are contained in the exhaust gas or further accurately detect the concentration of sulfur oxides in the exhaust gas.

A second aspect of the disclosure provides a control method for a gas detector. The gas detector includes an element, a voltage application device, a current detector, and an electronic control unit. The element is provided in an exhaust passage of an internal combustion engine. The element includes an electrochemical cell and a diffusion resistance body. The electrochemical cell includes a solid electrolyte body, a first electrode, and a second electrode. The solid electrolyte body has oxide ion conductivity. The first electrode and the second electrode are respectively provided on surfaces of the solid electrolyte body. The diffusion resistance body is formed of a porous material through which exhaust gas flowing through the exhaust passage can pass. The element is configured that the exhaust gas flowing through the exhaust passage reaches the first electrode through the diffusion resistance body. The voltage application device is configured to apply a voltage between the first electrode and the second electrode. The current detector is configured to detect an output current that is a current flowing between the first electrode and the second electrode. The control method includes: controlling an applied voltage that is the voltage applied between the first electrode and the second electrode by the voltage application device; either determining, by the electronic control unit, whether sulfur oxides in a predetermined concentration or higher are contained in the exhaust gas or detecting, by the electronic control unit, a concentration of the sulfur oxides in the exhaust gas, based on the output current detected by the current detector; when an air-fuel ratio of air mixture that is supplied to the internal combustion engine is in a stable state, executing, by the electronic control unit, boosting sweep for boosting the applied voltage from a predetermined voltage to a second voltage by the voltage application device and then executing, by the electronic control unit, lowering sweep for lowering the applied voltage from the second voltage to a first voltage at a predetermined lowering rate; obtaining, by the electronic control unit, a parameter that is correlated with a degree of a change occurred to the output current based on the output current detected by the current detector; either determining, by the electronic control unit, whether sulfur oxides in the predetermined concentration or higher are contained in the exhaust gas or detecting, by the electronic control unit, the concentration of the sulfur oxides in the exhaust gas based on the parameter; setting, by the electronic control unit, the predetermined lowering rate such that a rate of the reoxidation reaction becomes a rapidly increased rate at a time when the applied voltage becomes a voltage within a range between the first voltage and the decomposition initiation voltage of sulfur oxides; obtaining, by the electronic control unit, a value that is correlated with the output current in a predetermined period as a first current based on the output current detected by the current detector in the predetermined period; obtaining a second current by the electronic control unit; and calculating, by the electronic control unit, a difference between the obtained second current and the obtained first current and using the difference as the parameter. The predetermined voltage is a voltage that is equal to or higher than the first voltage and is lower than a decomposition initiation voltage of the sulfur oxides. The first voltage is a voltage that is lower than the decomposition initiation voltage of the sulfur oxides. The first voltage is a voltage when the output current becomes a limiting current of oxygen. The second voltage is a voltage that is higher than the decomposition initiation voltage of the sulfur oxides. The change occurred to the output current is a change in the output current resulted from a current flowing between the first electrode and the second electrode when a reoxidation reaction of sulfur that has been adsorbed to the first electrode leads to generation of the sulfur oxides in the first electrode when the applied voltage becomes lower than the decomposition initiation voltage of the sulfur oxides during the lowering sweep. The degree of the change occurred to the output current is increased as the concentration of the sulfur oxides contained in the exhaust gas is increased. The predetermined period is a period in which the lowering sweep is executed and in which the applied voltage is within a range between the first voltage and the decomposition initiation voltage of the sulfur oxides. The first voltage is excluded from the range. The decomposition initiation voltage is included in the range. The second current is the output current detected by the current detector at a time point at which the applied voltage is a particular voltage that is equal to or higher than a voltage at which the output current is the limiting current of oxygen. The second current is the output current that exclude a current resulted from the reoxidation reaction of sulfur that has been adsorbed to the first electrode in the first electrode by the boosting sweep.

### BRIEF DESCRIPTION OF THE DRAWINGS

Features, advantages, and technical and industrial significance of exemplary embodiments will be described below with reference to the accompanying drawings, in which like numerals denote like elements, and wherein:

FIG. 1 is a schematic configuration diagram of a gas detector according to a first embodiment of the disclosure and an internal combustion engine, to which the gas detector is applied;

FIG. 2 is a schematic cross-sectional view of one configuration example of an element of a gas sensor shown in FIG. 1;

FIG. 3A is a time chart that illustrates an overview of actuation of the gas detector according to the first embodiment;

FIG. 3B is a graph that indicates a waveform of an applied voltage during detection of SOx;

FIG. 3C is a graph that indicates another waveform of the applied voltage during the detection of SOx;

FIG. 4A is a schematic view that illustrates a SOx decomposition reaction occurred in the element;

FIG. 4B is a schematic view that illustrates a sulfur reoxidation reaction occurred in the element;

FIG. 5A is a graph that indicates a relationship between the applied voltage and an output current;

FIG. 5B is a graph that indicates a relationship between the applied voltage and the output current;

FIG. 6 is a graph that indicates a relationship between an A/F of air mixture in a combustion chamber and a limiting current range of oxygen;

FIG. 7 is a graph that indicates a relationship between an elapsed time and each of the applied voltage and the output current;

FIG. 8 is a flowchart of a sensor activation determination routine that is executed by a CPU of an ECU shown in FIG. 1;

FIG. 9 is a flowchart of an A/F detection routine that is executed by the CPU of the ECU shown in FIG. 1;

FIG. 10 is a flowchart of a SOx detection routine that is executed by the CPU of the ECU shown in FIG. 1;

FIG. 11 is a graph that indicates the relationship between the elapsed time and each of the applied voltage and the output current;

FIG. 12 is a flowchart of a SOx detection routine that is executed by a CPU of an ECU provided in a gas detector according to a second embodiment of the disclosure; and

FIG. 13 is a graph that indicates the relationship between the elapsed time and each of the applied voltage and the output current;

FIG. 14 is a flowchart of a SOx detection routine that is executed by a CPU of an ECU provided in a gas detector according to a third embodiment of the disclosure;

FIG. 15 is a flowchart of a SOx detection routine that is executed by a CPU of an ECU according to a modified example of the gas detector shown in FIG. 1;

FIG. 16 is a flowchart of a SOx detection routine that is executed by a CPU of an ECU according to another modified example of the gas detector shown in FIG. 1; and

FIG. 17 is a flowchart of a SOx detection routine that is executed by a CPU of an ECU according to yet another modified example of the gas detector shown in FIG. 1.

### DETAILED DESCRIPTION OF EMBODIMENTS

A description will hereinafter be made on a gas detector according to each embodiment of the disclosure with reference to the drawings. Note that the same or corresponding portions in all of the drawings of the embodiments are denoted by the same reference numerals.

### First Embodiment

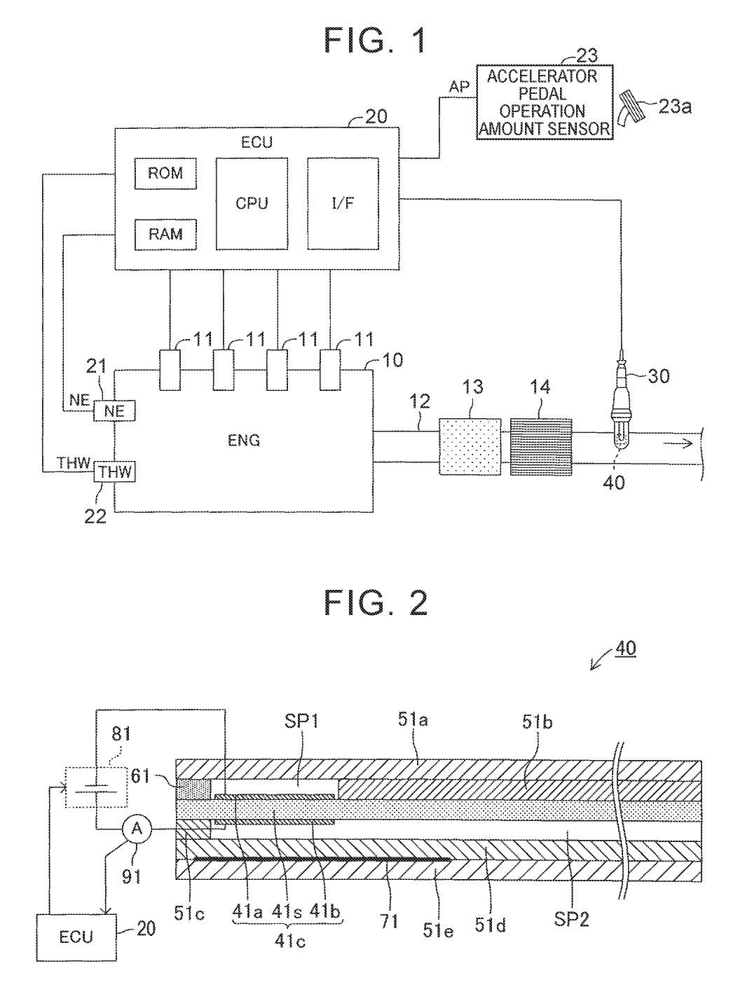

A description will be made on a gas detector according to a first embodiment of the disclosure (hereinafter may also be referred to as a “first detector”). The first detector is applied to a vehicle, which is not shown, and on which an internal combustion engine 10 shown in FIG. 1 is mounted.

The internal combustion engine 10 in this embodiment is a diesel engine. The internal combustion engine 10 includes a combustion chamber (not shown) and a fuel injection valve 11. The fuel injection valve 11 is disposed in a cylinder head section of the internal combustion engine 10, so as to be able to inject fuel into the combustion chamber. The fuel injection valve 11 directly injects the fuel into the combustion chamber in accordance with a command of an electronic control unit (ECU) 20, which will be described below. An exhaust pipe 12 is connected to an end of an exhaust manifold that is connected to an exhaust port communicating with the combustion chamber. The exhaust port and the exhaust manifold are not shown. The exhaust port, the exhaust manifold, and the exhaust pipe 12 constitute an exhaust passage, through which exhaust gas discharged from the combustion chamber flows. A diesel oxidation catalyst (DOC) 13 and a diesel particulate filter (DPF) 14 are disposed in the exhaust pipe 12.

The DOC 13 is an exhaust gas control catalyst. More specifically, the DOC 13 has precious metals such as platinum and palladium as catalysts, and oxidizes unburned components (HC, CO) in the exhaust gas to purify the exhaust gas. That is, by the DOC 13, oxidation of HC leads to generation of water and CO₂, and oxidation of CO leads to the generation of CO₂.

The DPF 14 is arranged on a downstream side of the DOC 13. The DPF 14 is a filter that catches particulate matters (PM) in the exhaust gas. More specifically, the DPF 14 includes plural passages, each of which is formed of a porous material (for example, a partition wall made of cordierite as one type of ceramic, for example). The DPF 14 collects the particulate matters, which are contained in the exhaust gas passing through the partition wall, in a pore surface of the partition wall.

The first detector includes the ECU 20. The ECU 20 is an electronic control circuit having a microcomputer, which includes a CPU, ROM, RAM, backup RAM, and an interface (IF), as a primary component. The CPU executes an instruction (a routine) stored in memory (the ROM) to realize a specified function.

The ECU 20 is connected to various actuators (the fuel injection valve 11 and the like) of the internal combustion engine 10. The ECU 20 sends a drive (command) signal to each of these actuators to control the internal combustion engine 10. Furthermore, the ECU 20 is connected to various types of sensors, which will be described below, and receives signals from these sensors.

An engine speed sensor (hereinafter referred to as an “NE sensor”) 21 measures a speed (an engine speed) NE of the internal combustion engine 10 and outputs a signal representing this engine speed NE.

A coolant temperature sensor 22 is disposed in a cylinder block section. The coolant temperature sensor 22 measures a temperature of a coolant (a coolant temperature TRW) that cools the internal combustion engine 10, and outputs a signal representing this coolant temperature TRW.

An accelerator pedal operation amount sensor 23 detects an operation amount of an accelerator pedal 23a of the vehicle and outputs a signal representing an accelerator pedal operation amount AP.

A gas sensor 30 is a limiting current type gas sensor of one cell type and is disposed in the exhaust pipe 12 that constitutes the exhaust passage of the engine 10.

The gas sensor 30 is disposed on a downstream side of the DOC 13 and the DPF 14 that are installed in the exhaust pipe 12.

Configuration of Gas Sensor

Next, a description will be made on a configuration of the gas sensor 30 with reference to FIG. 2. An element 40 that is provided in the gas sensor 30 includes a solid electrolyte body 41s, a first alumina layer 51a, a second alumina layer 51b, a third alumina layer 51c, a fourth alumina layer 51d, a fifth alumina layer 51e, a diffusion resistance body (a diffusion-controlled layer) 61, and a heater 71.

The solid electrolyte body 41s is a thin plate body that contains zirconia and the like and has oxide ion conductivity. Zirconia that forms the solid electrolyte body 41s may contain elements such as scandium (Sc) and yttrium (Y).

Each of the first to fifth alumina layers 51a to 51e is a dense (gas-impermeable) layer (a dense thin plate body) that contains alumina.

The diffusion resistance body 61 is a porous diffusion-controlled layer and is a gas-permeable layer (a thin plate body). The heater 71 is a thin cermet plate body that contains platinum (Pt) and ceramic (for example, alumina or the like), for example, and is a heat generation body that generates heat by energization. The heater 71 is connected to a power supply, which is not shown and is mounted on the vehicle, by lead wire, which is not shown. The heater 71 can change a heat generation amount when the ECU 20 controls an amount of power supplied from the power supply.

The layers of the element 40 are stacked in an order of the fifth alumina layer 51e, the fourth alumina layer 51d, the third alumina layer 51c, the solid electrolyte body 41s, the diffusion resistance body 61 and the second alumina layer 51b, and the first alumina layer 51a from below.

An internal space SP1 is a space that is formed by the first alumina layer 51a, the solid electrolyte body 41s, the diffusion resistance body 61, and the second alumina layer 51b, and the exhaust gas of the internal combustion engine 10 as detected gas is introduced thereinto via the diffusion resistance body 61. That is, the internal space SP1 communicates with the inside of the exhaust pipe 12 of the internal combustion engine 10 via the diffusion resistance body 61. Accordingly, the exhaust gas in the exhaust pipe 12 is led as the detected gas into the internal space SP1.

A first atmosphere intake passage SP2 is formed by the solid electrolyte body 41s, the third alumina layer 51c, and the fourth alumina layer 51d and is exposed to the atmosphere on the outside of the exhaust pipe 12.

A first electrode 41a is fixed to a surface on one side of the solid electrolyte body 41s. More specifically, the surface on the one side of the solid electrolyte body 41s is a surface of the solid electrolyte body 41s that defines the internal space SP1. The first electrode 41a is a negative electrode. The first electrode 41a is a porous cermet electrode that contains platinum (Pt) as a primary component.

A second electrode 41b is fixed to a surface on the other side of the solid electrolyte body 41s. More specifically, the surface on the other side of the solid electrolyte body 41s is a surface of the solid electrolyte body 41s that defines the first atmosphere intake passage SP2. The second electrode 41b is a positive electrode. The second electrode 41b is a porous cermet electrode that contains platinum (Pt) as a primary component.

The first electrode 41a and the second electrode 41b are arranged to oppose each other with the solid electrolyte body 41s being interposed therebetween. The first electrode 41a, the second electrode 41b, and the solid electrolyte body 41s constitute an electrochemical cell 41c that has oxygen discharging capacity realized by an oxygen pumping effect. The electrochemical cell 41c is heated to an activation temperature by the heater 71.

Each layer of the solid electrolyte body 41s and the first to fifth alumina layers 51a to 51e is molded in a sheet shape by a doctor blade method, an extrusion method, or the like, for example. The first electrode 41a, the second electrode 41b, wires used to energize these electrodes, and the like are each formed by a screen printing method, for example. These sheets are stacked as described above and are calcined. In this way, the element 40 with the structure as described above is integrally manufactured.

Note that the material constituting the first electrode 41a is not limited to the above material but can be selected from a material that contains a platinum group element such as platinum (Pt), rhodium (Rh), or palladium (Pd), an alloy thereof, or the like as a primary component. However, the material constituting the first electrode 41a is not particularly limited as long as SOx contained in the exhaust gas, which is led to the internal space SP1 via the diffusion resistance body 61, can be subjected to reductive decomposition when a voltage that is equal to or higher than a SOx decomposition initiation voltage (more specifically, a voltage of approximately 0.6 V or higher) is applied between the first electrode 41a and the second electrode 41b.

The gas sensor 30 further includes a power supply circuit 81 and an ammeter 91. The power supply circuit 81 and the ammeter 91 are connected to the above-described ECU 20.

The power supply circuit 81 can apply a predetermined voltage (hereinafter also referred to as an “applied voltage Vm”) between the first electrode 41a and the second electrode 41b such that an electric potential of the second electrode 41b is higher than an electric potential of the first electrode 41a. The power supply circuit 81 can change the applied voltage Vm when being controlled by the ECU 20. The power supply circuit 81 is one example of the voltage application device.

The ammeter 91 measures an output current (an electrode current) Im that is a current flowing between the first electrode 41a and the second electrode 41b (thus, a current flowing through the solid electrolyte body 41s), and outputs a measurement value to the ECU 20. The ammeter 91 is one example of the current detector.

Overview of Actuation

Next, a description will be made on an overview of actuation of the first detector. The first detector is configured to detect an oxygen concentration in the exhaust gas (the detected gas) that is discharged from the internal combustion engine 10. The first detector is configured to detect an air-fuel ratio (A/F) of air mixture in the combustion chamber of the internal combustion engine 10 on the basis of the oxygen concentration in the exhaust gas. The air-fuel ratio of the air mixture in the combustion chamber of the internal combustion engine 10 will hereinafter also be referred to as an “engine air-fuel ratio A/F”. Furthermore, the first detector is configured to determine presence or absence of SOx in a predetermined concentration or higher that is contained in the exhaust gas. Because several seconds are required from initiation of detection of the presence or the absence of SOx to termination of the detection thereof, the first detector is configured to determine the presence or the absence of SOx in the predetermined concentration or higher in a state where the engine air-fuel ratio A/F is stable.

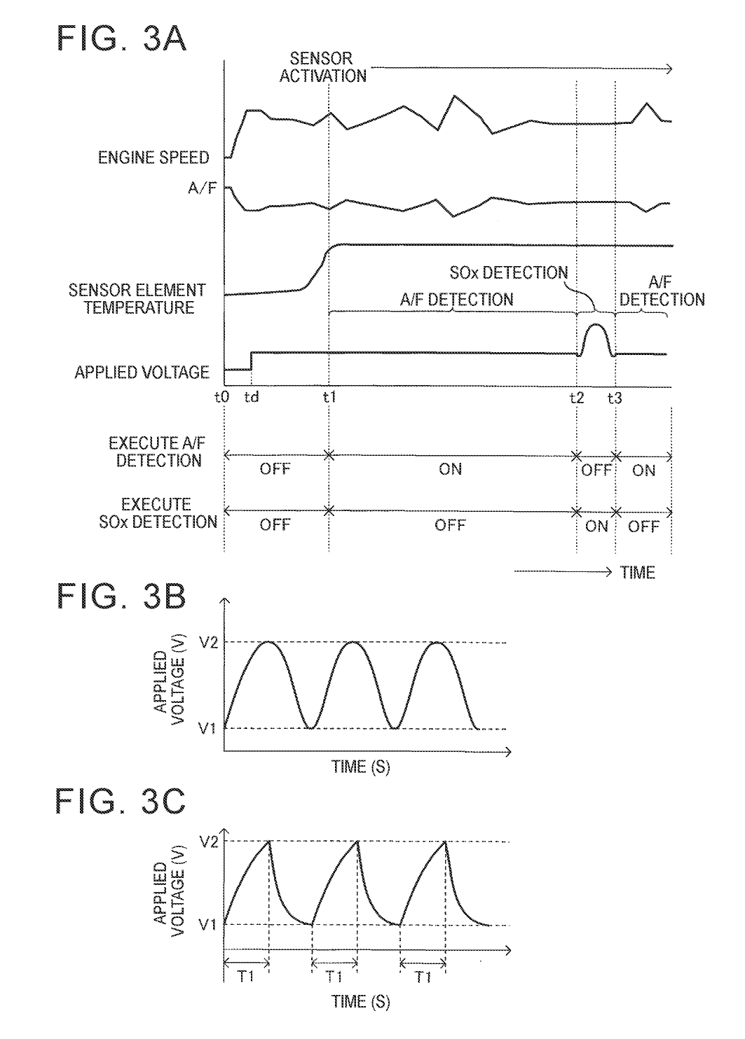

More specifically, as shown in FIG. 3A, at time t0 as a time point at which the internal combustion engine 10 is started, the first detector starts controlling the heater 71 such that the solid electrolyte body 41s is heated by the heater 71. In this way, a temperature of the solid electrolyte body 41s is increased to a predetermined temperature that is equal to or higher than a temperature at which the oxide ion conductivity appears (hereinafter may also be referred to as the “activation temperature”).

At time t1, the temperature of the solid electrolyte body 41s (a sensor element temperature) becomes equal to or higher than the activation temperature, and the gas sensor 30 is brought into a sensor active state. At this time, the first detector starts processing to detect the oxygen concentration in the exhaust gas and obtain the engine air-fuel ratio A/F on the basis of the oxygen concentration. Note that, at time td as a time point between the time t0 and the time t1, the first detector starts applying an oxygen concentration (A/F) detection voltage (more specifically, 0.4 V), which is appropriate for the detection of the oxygen concentration, between the first electrode 41a and the second electrode 41b. That is, the first detector sets the applied voltage Vm to the oxygen concentration detection voltage. In the case where this applied voltage Vm is set to the oxygen concentration detection voltage when the temperature of the solid electrolyte body 41s is equal to or higher than the activation temperature, oxygen molecules are decomposed, and the oxygen pumping effect appears. In this case, gas of oxygen containing components (including SOx) other than oxygen is not decomposed. Because the oxygen concentration detection voltage is lower than decomposition initiation voltages of the oxygen containing components (including SOx) other than oxygen, the oxygen containing components other than oxygen are not decomposed.

The first detector successively detects the oxygen concentration from the time t1 and thereby monitors the engine air-fuel ratio A/F. Then, when a SOx detection initiation condition is satisfied (that is, when the engine air-fuel ratio A/F is brought into a stable state and the other conditions, which will be described below, are satisfied) at the time t2, the first detector starts the processing to detect the SOx concentration in the exhaust gas. That is, in a period from the time t1 to time immediately before the time t2, the first detector detects the engine air-fuel ratio A/F. At the time t2 as a time point of starting the detection of the SOx concentration, the first detector stops detecting the engine air-fuel ratio A/F.

Note that, in this specification, the detection of the SOx concentration indicates both of the detection (measurement) of the SOx concentration itself in the exhaust gas and obtainment of a parameter that represents the SOx concentration in the exhaust gas. As will be described below, this detector obtains the parameter that represents the SOx concentration in the exhaust gas (a parameter that varies in accordance with the SOx concentration), and uses the parameter to determine whether SOx in the predetermined concentration or higher is contained in the exhaust gas. As the predetermined concentration, a concentration that is higher than 0 and that corresponds to a desired detection level is selected.

In a period from the time t2 to time immediately before time t3, the first detector executes applied voltage sweep within a predetermined applied voltage range (an applied voltage sweep range between a lower limit voltage (a first voltage V1) and an upper limit voltage (a second voltage V2)). More specifically, after executing boosting sweep for gradually boosting the applied voltage Vm from the first voltage V1 to the second voltage V2, the first detector executes lowering sweep for gradually lowering the applied voltage Vm from the second voltage V2 to the first voltage V1. The first detector executes one cycle of the applied voltage sweep that includes one time of the boosting sweep and one time of the lowering sweep as one cycle. However, the first detector may execute the plural cycles of the applied voltage sweep.

Note that, when the applied voltage for the detection of the oxygen concentration (the A/F) is higher than the first voltage V1, the first detector may start the first boosting sweep by using the applied voltage for the detection the oxygen concentration as the applied voltage Vm. Alternatively, when the applied voltage for the detection of the oxygen concentration is higher than the first voltage V1, the first detector may lower the applied voltage Vm from the applied voltage for the detection of the oxygen concentration to the first voltage V1 and then start the first boosting sweep.

More specifically, as shown in FIG. 3B, the first detector executes the applied voltage sweep by applying the voltage with a sine waveform (of one cycle) between the first electrode 41a and the second electrode 41b. Note that the voltage waveform in this case is not limited to the sine wave shown in FIG. 3B and any of various waveforms can be adopted therefor. For example, the voltage waveform in this case may be a non-sine wave as indicated in a graph of FIG. 3C (a waveform such as the voltage waveform during charging/discharging of a capacitor).

When terminating the detection of SOx at the time t3, the first detector resumes the processing to detect the engine air-fuel ratio A/F. That is, the first detector sets the applied voltage Vm to the oxygen concentration detection voltage (0.4 V) at the time t3.

Detection of A/F

Next, a description will be made on the actuation of the first detector at a time when the first detector detects the above-described engine air-fuel ratio A/F. When the gas sensor 30 is brought into the sensor active state, in order to obtain the engine air-fuel ratio A/F, the first detector sets the applied voltage Vm to the oxygen concentration detection voltage (for example, 0.4 V) such that the first electrode 41a has the low electric potential and the second electrode 41b has the high electric potential. That is, the first electrode 41a functions as the negative electrode, and the second electrode 41b functions as the positive electrode. The oxygen concentration detection voltage is set to a voltage that is equal to or higher than a voltage (the decomposition initiation voltage) at which the decomposition of oxygen (O₂) is started in the first electrode 41a and that is lower than the decomposition initiation voltages of the oxygen containing components other than oxygen. In this way, oxygen contained in the exhaust gas is subjected to the reductive decomposition in the first electrode 41a, which leads to generation of oxide ions (O²⁻).

These oxide ions are conducted to the second electrode 41b via the above solid electrolyte body 41s, become oxygen (O₂), and is discharged to the atmosphere through the first atmosphere intake passage SP2. As described above, such movement of oxygen by the conduction of the oxide ions from the negative electrode (the first electrode 41a) to the positive electrode (the second electrode 41b) via the solid electrolyte body 41s is referred to as the oxygen pumping effect.

Due to the conduction of the oxide ions associated with this oxygen pumping effect, the current flows between the first electrode 41a and the second electrode 41b. The current that flows between the first electrode 41a and the second electrode 41b is referred to as the “output current Im (or the electrode current Im)”. In general, the output current Im has a tendency of being increased as the applied voltage Vm is boosted. However, because a flow rate of the exhaust gas that reaches the first electrode 41a is restricted by the diffusion resistance body 61, an oxygen consumption rate that is associated with the oxygen pumping effect eventually exceeds an oxygen supply rate to the first electrode 41a. That is, an oxygen reductive decomposition reaction in the first electrode 41a (the negative electrode) is brought into a diffusion-controlled state.

Once the oxygen reductive decomposition reaction in the first electrode 41a is brought into the diffusion-controlled state, the output current Im is not increased even when the applied voltage Vm is boosted, and remains to be substantially constant. Such a characteristic is referred to as a “limiting current characteristic”. A range of the applied voltage where the limiting current characteristic appears (is observed) is referred to as a “limiting current range”. Furthermore, the output current Im within the limiting current range is referred to as a “limiting current”. A magnitude of the limiting current (a limiting current value) for oxygen corresponds to the oxygen supply rate to the first electrode 41a (the negative electrode). As described above, because the flow rate of the exhaust gas that reaches the first electrode 41a is maintained to be constant by the diffusion resistance body 61, the oxygen supply rate to the first electrode 41a corresponds to the concentration of oxygen contained in the exhaust gas.

Accordingly, in the gas sensor 30, the output current (the limiting current) Im at the time when the applied voltage Vm is set to a predetermined voltage within the limiting current range of oxygen (the oxygen concentration detection voltage, and more specifically, 0.4 V) corresponds to the concentration of oxygen contained in the exhaust gas. By using the limiting current characteristic of oxygen, just as described, the first detector detects the concentration of oxygen contained in the exhaust gas as the detected gas. Meanwhile, the engine air-fuel ratio A/F and the oxygen concentration in the exhaust gas establish a one-on-one relationship. Accordingly, the first detector stores this relationship in the ROM in advance and obtains the engine air-fuel ratio A/F on the basis of this relationship and the detected oxygen concentration. Note that the first detector may store a relationship between the limiting current of oxygen and the engine air-fuel ratio A/F in the ROM in advance and may obtain the engine air-fuel ratio A/F on the basis of the relationship and the limiting current of oxygen.

Detection of SOx Concentration

### Principle of Detection

Next, a description will be made on a method for detecting the SOx concentration in the exhaust gas. The above-described oxygen pumping effect is also exhibited by the oxygen containing components, such as SOx (sulfur oxides) and H₂O (water), that contain oxygen atoms in the molecules. That is, when a voltage that is equal to or higher than the decomposition initiation voltage of each of these compounds is applied between the first electrode 41a and the second electrode 41b, each of these compounds is subjected to the reductive decomposition, which leads to the generation of the oxide ions. These oxide ions are conducted from the first electrode 41a to the second electrode 41b by the oxygen pumping effect. In this way, the output current Im flows between the first electrode 41a and the second electrode 41b.

However, the concentration of SOx that is contained in the exhaust gas is extremely low, and thus the current resulted from the decomposition of SOx is extremely small. Furthermore, the current resulted from the decomposition of each of the oxygen containing components other than SOx (for example, water, carbon dioxide, and the like) also flows between the first electrode 41a and the second electrode 41b. Thus, it is difficult to accurately detect only the output current resulted from SOx.

In view of the above, as the result of the earnest investigation, the inventor of the present application has reached findings that the SOx concentration can accurately be detected by executing the applied voltage sweep that has the boosting sweep and the lowering sweep at a predetermined sweeping rate as one cycle at a time when the SOx concentration is detected.

The boosting sweep is processing to gradually boost the applied voltage Vm from the first voltage V1 to the second voltage V2. The lowering sweep is processing to gradually lower the applied voltage Vm from the second voltage V2 to the first voltage V1. Note that the first voltage V1 and the second voltage V2 correspond to the electric potential of the second electrode 41b with the electric potential of the first electrode 41a being a reference, and each have a positive voltage value.

The first voltage V1 is set to a voltage within a voltage range (hereinafter referred to as a “first voltage range”) that is lower than the SOx decomposition initiation voltage (approximately 0.6 V) and that is higher than a minimum value of the applied voltage Vm within the limiting current range of oxygen. Because the minimum value of the applied voltage Vm within the limiting current range of oxygen depends on the engine air-fuel ratio A/F, a lower limit value of the first voltage range is also desirably changed in accordance with the engine air-fuel ratio A/F. More specifically, the lower limit value of the first voltage range is a voltage within a range from 0.2 V to 0.45 V, for example, and an upper limit voltage of the first voltage range is 0.6 V. That is, the first voltage V1 is a voltage that is selected from a range between 0.2V and 0.6 V (the range includes 0.2 V and excludes 0.6 V).

The second voltage V2 is set to a voltage within a voltage range (hereinafter also referred to as a “second voltage range”) that is higher than the SOx decomposition initiation voltage (approximately 0.6 V) and that is lower than an upper limit value (2.0 V) of the voltage, at which the solid electrolyte body 41s is not damaged. That is, the second voltage V2 is a voltage that is selected from a range between 0.6 V and 2.0 V (the range excludes 0.6V and includes 2.0V).

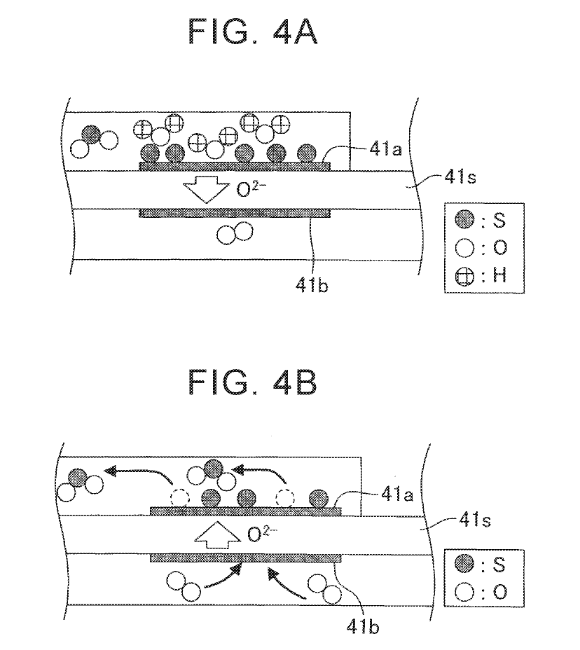

In a period in which the boosting sweep is executed, when the applied voltage Vm, which is applied between the first electrode 41a and the second electrode 41b, becomes equal to or higher than the SOx decomposition initiation voltage, as shown in FIG. 4A, the reductive decomposition of SOx contained in the exhaust gas leads to the generation of S and O²⁻ in the first electrode 41a (the negative electrode). As a result, a reductive decomposition product (S (sulfur)) of SOx is adsorbed to the first electrode 41a (the negative electrode).

In a period in which the lowering sweep is executed, when the applied voltage Vm becomes lower than the SOx decomposition initiation voltage, as shown in FIG. 4B, such a reaction that S, which has been adsorbed to the first electrode 41a (the negative electrode), and O²⁻ are reacted to generate SOx occurs (hereinafter this reaction may also be referred to as a S (sulfur) reoxidation reaction). At this time, the output current Im is changed as will be described below as a result of the S reoxidation reaction. Note that this change in the output current Im, which is associated with the “S reoxidation reaction”, is referred to as a “reoxidation current change”.

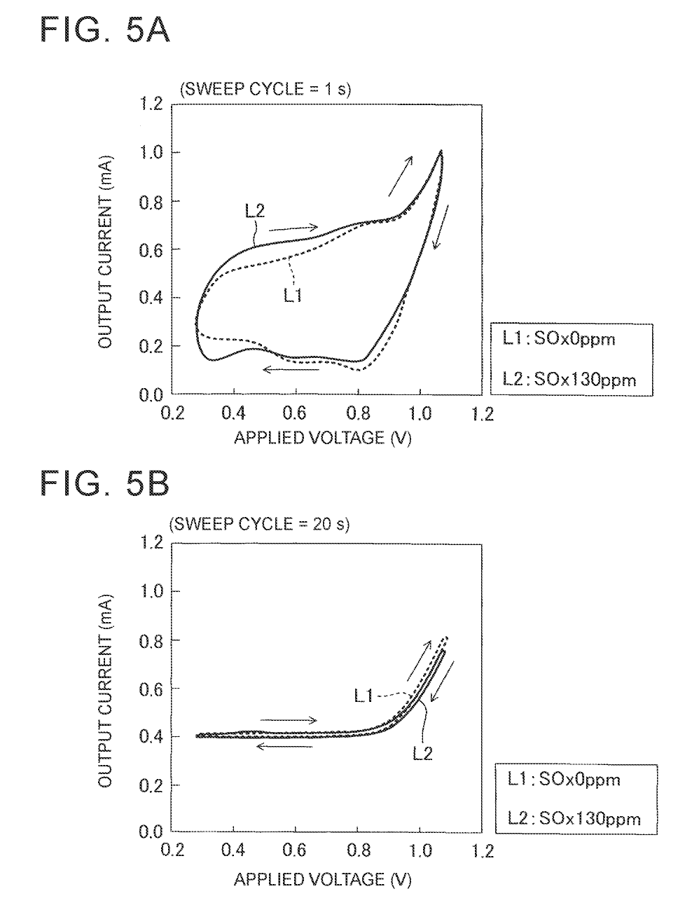

By the way, it has been found in the investigation by the inventor that the reoxidation current change, which yields a significant effect on the detection of the SOx concentration, does not always appear depending on the sweeping rate (a voltage lowering amount per predetermined elapsed time) in the lowering sweep. A description will be made on this point with reference to FIG. 5A and FIG. 5B.

FIG. 5A is a schematic graph of a relationship between the applied voltage Vm and the output current Im at a time when a sweep cycle (that is, a sum of a time required for the boosting sweep and a time required for the lowering sweep, the cycle of the applied voltage sweep) is set to one second and the applied voltage sweep is executed. FIG. 5B is a schematic graph of a relationship between the applied voltage Vm and the output current Im at a time when the applied voltage sweep is executed at the slower sweeping rate (the sweep cycle of 20 seconds) than that in the example shown in FIG. 5A.

When both of the graphs are compared, compared to the example in FIG. 5B, a difference between the “output current Im at a time when the SOx concentration of the detected gas is 0 ppm”, which is represented by a line L1, and the “output current Im at a time when the SOx concentration of the detected gas is 130 ppm”, which is represented by a line L2, (a difference in the current value) is clearly appeared within the voltage range of less than the SOx decomposition initiation voltage (0.6 V) in the example of FIG. 5A, in which the sweeping rate in the applied voltage sweep is higher than the example of FIG. 5B. That is, the current change (the reoxidation current change) that yields the significant effect on the detection of the SOx concentration appears in the example of FIG. 5A. A mechanism of causing such a phenomenon is considered as follows.

That is, in the case where the sweeping rate is decreased to be lower than a predetermined rate, the S reoxidation reaction is continuously and gradually progressed during the lowering sweep. Thus, the reoxidation current change, which yields the significant effect on the detection of the SOx concentration, does not appear. On the other hand, in the case where the sweeping rate is increased to be higher than the predetermined rate, the applied voltage Vm is lowered while the S reoxidation reaction is not significantly progressed during the lowering sweep. Then, it is considered that, once the applied voltage Vm becomes a voltage within a “certain voltage range where the S reoxidation reaction is activated”, the S reoxidation reaction is rapidly progressed. In this way, the current change that yields the significant effect on the detection of the SOx concentration appears.

Just as described, depending on the sweeping rate during the lowering sweep, a case where the current change that yields the significant effect on the detection of the SOx concentration appears and a case where the current change that yields the significant effect on the detection of the SOx concentration does not appear occur. Accordingly, when the lowering sweep is executed, it is required to set the sweeping rate to the predetermined rate at which the current change yielding the significant effect on the detection of the SOx concentration appears, and such a current change represents the reoxidation current change.

In the first detector, this predetermined rate is set to an appropriate rate, at which the current change yielding the significant effect on the detection of the SOx concentration appears, by an experiment in advance, and such a current change represents the reoxidation current change.

According to the experiment, it has been found that, when the voltage in the sine waveform shown in FIG. 3B is applied between the first electrode 41a and the second electrode 41b, for example, this predetermined rate may be set to a sweeping rate at a frequency F within a predetermined range (typically, a range between 0.1 Hz and 5 Hz inclusive). A lower limit value of the frequency F within this predetermined range is defined from such a perspective that a signal difference yielding the significant effect on the detection of the SOx concentration (the reoxidation current change) can no longer be obtained when the frequency F is further lowered. Meanwhile, an upper limit value of the frequency F within this predetermined range is defined from such a perspective that the frequency F further contributes to causes of the current change other than the SOx concentration (more specifically, capacity of the solid electrolyte body 41s, and the like) when the frequency F is further increased.

Meanwhile, according to the experiment, it has been found that, when the voltage in the non-sine waveform, which is associated with charging/discharging of the capacitor, as shown in FIG. 3C is applied between the first electrode 41a and the second electrode 41b, this predetermined rate may be set to such a sweeping rate that a response time T1 of a voltage switching waveform is within a predetermined range (typically, a range between 0.1 second and 5 seconds inclusive). Note that the response time T1 corresponds to a time required for the applied voltage Vm to be changed from a lower limit voltage to an upper limit voltage within a predetermined range and vice versa. The lower limit voltage and the upper limit voltage within the predetermined range of the response time T1 are set to appropriate values from a similar perspective to those in the case where the frequency F (the above predetermined frequency) is determined when the voltage in the above-described sine waveform is used as the applied voltage Vm.

Note that, when the predetermined ranges of the frequency F and the response time T1 described above are each converted to a required time for the lowering sweep (that is, a time required for the applied voltage Vm to reach the first voltage V1 from the second voltage V2), each of the predetermined ranges becomes a range between 0.1 second and 5 seconds inclusive. Thus, the time may fall within the range between 0.1 second and 5 seconds inclusive.

Furthermore, it has been found that the reoxidation current change primarily depends on the SOx concentration in the exhaust gas as the detected gas. In other words, there is a low possibility that the reoxidation current change is influenced by the gas of the oxygen containing components (for example, water) other than sulfur oxides (SOx) in the exhaust gas. That is, when the boosting sweep is executed, decomposed matters (for example, hydrogen as a decomposed matter of water, and the like) of the components (the oxygen containing components) other than sulfur oxides are not adsorbed to the first electrode 41a. Accordingly, in the period in which the lowering sweep is executed, such a phenomenon that such decomposed matters of the oxygen containing components other than sulfur oxides are subjected to the reoxidation reaction in the first electrode 41a and again become the oxygen containing components does not substantially occur.

Thus, the change in the output current that occurs when the reoxidation reaction of sulfur, which has been adsorbed to the first electrode 41a, in the first electrode 41a leads to the generation of sulfur oxides during the lowering sweep is less likely to be influenced by the oxygen containing components other than sulfur oxides. That is, the change in the output current that is less likely to be influenced by the oxygen containing components other than sulfur oxides occurs.

Furthermore, it has been found that the change in the output current (the reoxidation current change) appears to have such a characteristic that the output current Im is decreased as the SOx concentration in the exhaust gas (the detected gas) is increased. That is, when the sulfur reoxidation reaction occurs, as shown in FIG. 4B, the oxide ions are consumed in the first electrode 41a. Thus, an amount of movement of the oxide ions (for example, the oxide ions produced by the decomposition of the oxygen molecules) that move from the first electrode 41a to the second electrode 41b is decreased. In this way, the output current Im is decreased. As the SOx concentration in the exhaust gas is increased, an amount of sulfur that is adsorbed to the first electrode 41a particularly during the boosting sweep is increased. Accordingly, an amount of the oxide ions that is consumed by the reaction with sulfur in the first electrode 41a particularly during the lowering sweep is also increased. As a result, the amount of the oxide ions that move from the first electrode 41a to the second electrode 41h is decreased. Thus, as the SOx concentration in the exhaust gas is increased, the output current Im is decreased.

It is understood from what has been described so far that, when the above-described reoxidation current change is used, the SOx concentration in the exhaust gas can accurately be detected without being influenced by the gas of the oxygen containing components (for example, water) other than SOx in the exhaust gas. Accordingly, the first detector detects the SOx concentration (actually, determines the presence or the absence of SOx in the predetermined concentration or higher) by using this reoxidation current change.

Parameter For Detecting Reoxidation Current Change

The first detector obtains a parameter that appropriately (accurately) represents the reoxidation current change, and detects the SOx concentration on the basis of this parameter. More specifically, the first detector obtains a minimum value of the output current Im in the period in which the lowering sweep is executed and in which the applied voltage Vm is within a range (a detection voltage range) between a fourth voltage V4 and a current obtainment initiation voltage (a third voltage) Vsem inclusive, and obtains this minimum value of the output current Im as a value that is correlated with the output current Im in the above period (that is, a first current Ig). The fourth voltage V4 is higher than the first voltage V1.

The current obtainment initiation voltage Vsem is selected from a range between the lower limit voltage (the first voltage V1) of the lowering sweep and the SOx decomposition initiation voltage (0.6 V), the lower limit voltage is excluded from the range, and the SOx decomposition initiation voltage is included in the range. In this example, the current obtainment initiation voltage Vsem is set at 0.6 V. Note that the current obtainment initiation voltage Vsem may differ in accordance with at least one of the applied voltage range of the applied voltage sweep and the cycle of the applied voltage sweep (in other words, the sweeping rate in the applied voltage sweep). The current obtainment initiation voltage Vsem may be higher than the lower limit voltage (the first voltage V1) of the voltage range in the applied voltage sweep, may be lower than the SOx decomposition initiation voltage (0.6 V), and may be higher than the first voltage V1 and equal to or lower than 0.45 V.

Furthermore, the first detector obtains the output current Im at a time when the applied voltage Vm is the voltage for the detection of the engine air-fuel ratio A/F, and obtains this output current Im as a second current Ib. Moreover, the first detector obtains a difference Idiff (=Ib−Ig) that is obtained by subtracting the first current Ig from the second current Ib as the parameter that represents the reoxidation current change. Then, the first detector detects the SOx concentration (actually, determines the presence or the absence of SOx in the predetermined concentration or higher) on the basis of this parameter (the difference Idiff).

The first detector obtains the difference Idiff by executing the only one cycle of the applied voltage sweep but may be configured as follows. More specifically, the first detector may be configured to execute the plural cycles of the applied voltage sweep, obtain the difference Idiff in each of the cycles, and uses an average value of the obtained “differences Idiff” as the parameter that represents the reoxidation current change.

Method for Detecting SOx Concentration

The first detector detects the SOx concentration (actually determines the presence or the absence of SOx in the predetermined concentration or higher) as follows by using the detection principle of the SOx concentration that has been described so far.

The first detector executes the applied voltage sweep at the predetermined sweeping rate. In this case, what is especially important is a lowering sweeping rate. At this time, the first detector determines the voltage range (that is, the first voltage V1 and the second voltage V2) of the applied voltage sweep on the basis of the engine air-fuel ratio A/F that is detected by using the oxygen concentration in the exhaust gas obtained immediately before the determination.

The first detector obtains the output current Im of the applied voltage (0.4 V) during the detection of the A/F as the second current Ib.

The first detector obtains the minimum value of the output current Im at the time when the applied voltage Vm is within the detection voltage range (the range between the first voltage V1 and the current obtainment initiation voltage Vsem, the first voltage V1 is excluded from the range, and the current obtainment initiation voltage is included in the rage) during the lowering sweep, and obtains this minimum value of the output current Im as the first current Ig.

The first detector calculates the difference Idiff (=Ib−Ig) obtained by subtracting the first current Ig from the second current Ib. This difference Idiff is the parameter that represents the SOx concentration in the exhaust gas.

The first detector determines whether SOx in the predetermined concentration or higher is contained in the exhaust gas on the basis of the difference Idiff.

More specifically, when executing the applied voltage sweep for the detection of the SOx concentration, the first detector applies the one cycle of the voltage in the sine waveform shown in FIG. 3B between the first electrode 41a and the second electrode 41b. At this time, the first detector executes the applied voltage sweep (the boosting sweep and the lowering sweep) at the above-described “predetermined sweeping rate”, at which the current change yielding the significant effect on the already-described detection of the SOx concentration occurs.

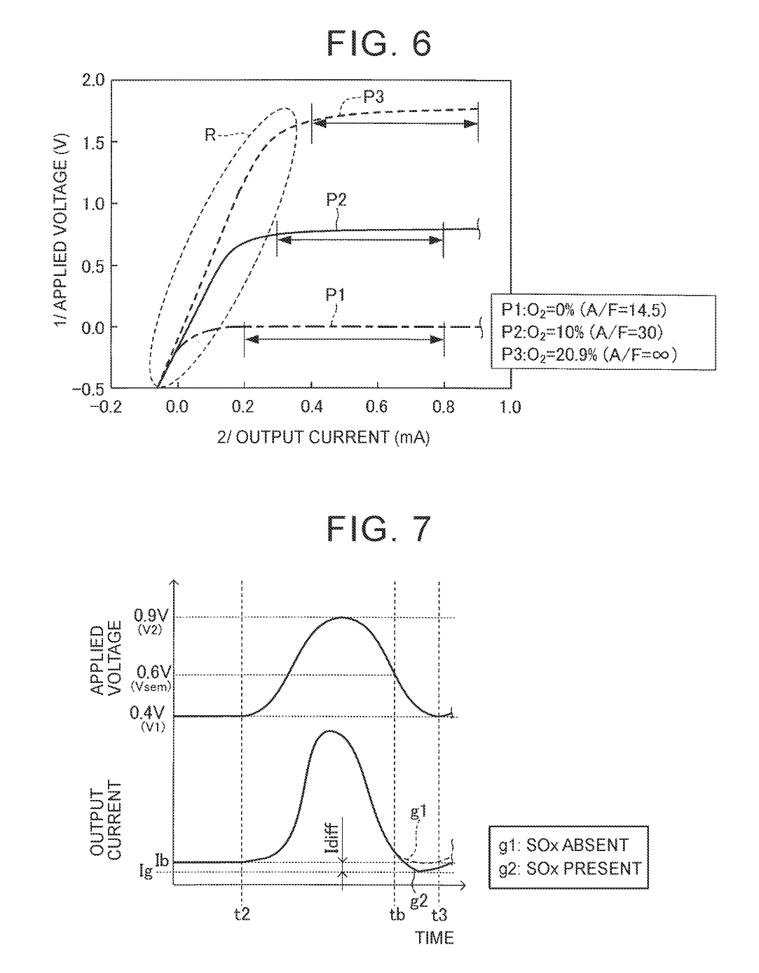

At this time, the first detector determines the voltage range of the applied voltage sweep (the lower limit voltage (the first voltage V1) and the upper limit voltage (the second voltage V2) of the applied voltage sweep) on the basis of the engine air-fuel ratio A/F. More specifically, as shown in FIG. 6, the lower limit voltage of the applied voltage sweep is defined to avoid the detection of the output current Im that is within an internal resistance dependence range surrounded by a dotted line R. This internal resistance dependence range is a region in which the output current Im is increased along with boosting of the applied voltage Vm (a region immediately before the output current Im reaches the limiting current range of oxygen). The upper limit value of the applied voltage Vm within the internal resistance dependence range (that is, the minimum value of the applied voltage within the limiting current range of oxygen) is gradually boosted as the engine air-fuel ratio A/F becomes leaner (the oxygen concentration in the exhaust gas is increased). While the upper limit voltage of the applied voltage sweep may be constant, the lower limit voltage (the first voltage V1) of the applied voltage sweep is defined to be boosted as the engine air-fuel ratio A/F becomes leaner.

More specifically, the upper limit value of the applied voltage Vm within the internal resistance dependence range R is increased as the engine air-fuel ratio A/F becomes leaner. Accordingly, the first detector boosts the lower limit voltage (the first voltage V1) of the applied voltage sweep as the engine air-fuel ratio A/F becomes leaner so that the voltage range of the applied voltage sweep does not enter this internal resistance dependence range R.

According to the experiment by the inventor, when A/F=14.5 (stoichiometric), the first voltage V1 may have a value that is selected from 0.2 V or higher, and the first detector sets the first voltage V1 at 0.2 V. When A/F=30, the first voltage V1 may have a value that is selected from 0.3 V or higher, and the first detector sets the first voltage V1 at 0.3 V. When A/F=is infinity (the O₂ concentration=20.9%), the first voltage V1 may have a value that is selected from 0.4 V or higher, and the first detector sets the first voltage V1 at 0.4 V.

As it has already been described, in the case where SOx is contained in the exhaust gas when the boosting sweep and the lowering sweep are executed, S (sulfur), which is produced by the decomposition of SOx is adsorbed to the first electrode 41a in the period during the boosting sweep. In the period during the lowering sweep, S that has been adsorbed to the first electrode 41a is reoxidized.

The first detector detects the reoxidation current change by using the above-described parameter (the difference Idiff) and thereby detects the SOx concentration (actually, determines the presence or the absence of SOx in the predetermined concentration or higher).

More specifically, as shown in FIG. 7, the first detector sets the applied voltage Vm to the applied voltage (0.4 V) during the A/F detection at time before the time point (the time t2) at which the applied voltage sweep is initiated, and obtains the output current Im at the time as the second current Ib. Furthermore, the first detector obtains the minimum value of the output current Im (the output current Im indicated by a line g2) in a period (a period from time tb to the time t3) in which the lowering sweep is executed and in which the applied voltage Vm is within the range (that is, the detection voltage range) between the fourth voltage V4, which is higher than the first voltage V1, and the current obtainment initiation voltage Vsem (0.6V) inclusive, and obtains this minimum value of the output current Im as the first current Ig. Moreover, the first detector calculates the difference Idiff (=Ib−Ig) that is obtained by subtracting the first current Ig from the second current Ib. The first detector detects the SOx concentration (actually, determines the presence or the absence of SOx in the predetermined concentration or higher) on the basis of the difference Idiff.

As indicated by the line g2, when SOx is contained in the exhaust gas, the output current Im (the second current Ib) in the period (the period from the time tb to the time t3), in which the applied voltage Vm is the voltage within the detection voltage range during the lowering sweep, shows the following behavior. More specifically, such a “degree of the reoxidation current change” appears that the output current Im of the case that is indicated by the line g2 and where SOx is contained in the exhaust gas is smaller than the output current Im of the case that is indicated by a line g1 and where SOx is not contained in the exhaust gas. Accordingly, the minimum value (the first current Ig) of the output current Im in the above period has a characteristic of being smaller than the minimum value (a current Ir) of the output current Im of the case where SOx is not contained in the exhaust gas. Furthermore, this first current Ig has a characteristic of being decreased as the SOx concentration is increased.

In addition, the output current Im in the period (the period from the time tb to the time t3) in which the applied voltage Vm is the voltage within the detection voltage range during the lowering sweep is changed under the influence of the oxygen concentration in the exhaust gas. More specifically, this output current Im is increased as the oxygen concentration in the exhaust gas is increased (as the engine air-fuel ratio A/F becomes leaner). Accordingly, the first current Ig is increased as the oxygen concentration in the exhaust gas is increased.

Meanwhile, the output current Im in a period in which the applied voltage sweep is not executed and in which the applied voltage Vm is set to have a constant value is more stable than the output current Im during the applied voltage sweep. Furthermore, when the applied voltage Vm is set to the “applied voltage (the applied voltage for the detection of the oxygen concentration that is within the limiting current range of oxygen) during the detection of A/F that is lower than the SOx decomposition initiation voltage (approximately 0.6 V)”, the output current Im has a value that corresponds to the oxygen concentration in the exhaust gas. In addition, the output current Im (the second current Ib) at the time (immediately before the time t2) before the applied voltage sweep at which the applied voltage Vm is set to the applied voltage during the A/F detection is not changed by the SOx concentration in the exhaust gas, and a magnitude of the second current Ib of the case where SOx is contained in the exhaust gas is equal to the magnitude of the second current Ib of the case where SOx is not contained in the exhaust gas.

Because the first current Ig and the second current Ib have the characteristics that have been described so far, the difference Idiff (=Ib−Ig) of the case where SOx is contained in the exhaust gas is larger than a difference Ir (=Ib−Ir) of the case where SOx is not contained in the exhaust gas. Furthermore, while the magnitude of the second current Ib remains the same regardless of the SOx concentration, the degree of the reoxidation current change becomes significant, and the first current Ig is thereby decreased as the SOx concentration is increased. Thus, the difference Idiff is also increased as the SOx concentration is increased. In addition, the first current Ig is changed under the influence of the oxygen concentration in the exhaust gas, and a degree of the change thereof appears in the second current Ib. Accordingly, the difference Idiff is not influenced by the oxygen concentration in the exhaust gas (the engine air-fuel ratio A/F) and is the parameter that accurately represents the concentration of sulfur oxides. As a result of this, the first detector can accurately determine whether SOx in the predetermined concentration or higher exists in the exhaust gas on the basis of this parameter (the difference Idiff).

Specific Actuation

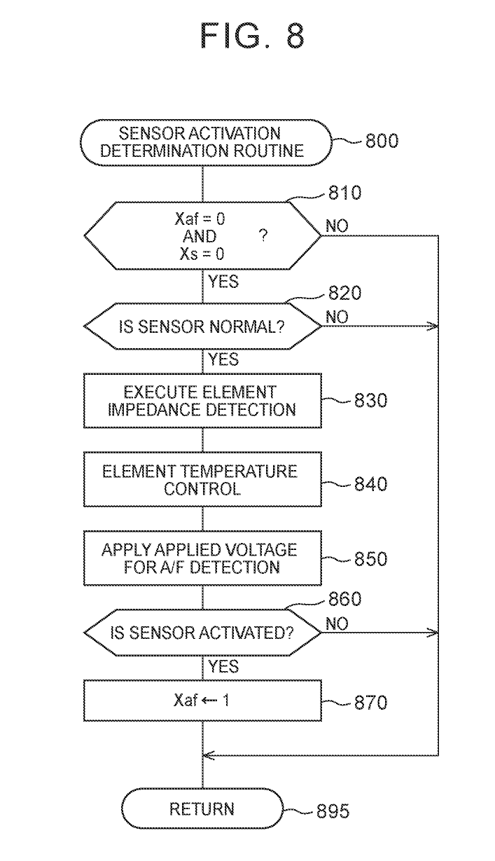

Next, a description will be made on specific actuation of the first detector. Every time predetermined time elapses, the CPU of the ECU 20 (hereinafter simply referred to as the “CPU”) uses the gas sensor 30 to execute a sensor activation determination routine, an A/F detection routine, and a SOx detection routine that are respectively shown in flowcharts of FIG. 8 to FIG. 10.

Note that a value of an A/F detection request flag Xaf and a value of a SOx detection request flag Xs that are used in these routines are set to “0” in an initial routine executed by the CPU when an ignition key switch, which is not shown and is mounted on the vehicle, is switched from an OFF position to an ON position.

At predetermined timing, the CPU starts processing from step 800 of the sensor activation determination routine shown in FIG. 8. Then, the processing proceeds to step 810, and the CPU determines whether both of the value of the A/F detection request flag Xaf and the value of the SOx detection request flag Xs are “0”.

If a current time point is a time point immediately after the ignition key switch is switched to the ON position (immediately after the internal combustion engine 10 is started), both of the value of the A/F detection request flag Xaf and the value of the SOx detection request flag Xs are “0”. Accordingly, the CPU determines Yes in step 810, and the processing proceeds to step 820. Then, the CPU determines whether the gas sensor 30 is normal by a well-known method. For example, the CPU determines that the gas sensor 30 is abnormal in the cases where the A/F is detected during the last operation of the internal combustion engine 10 and the output current Im is not changed when an operation state of the internal combustion engine 10 is changed from a fuel injection state to a fuel cut state. Then, the CPU stores the determination in the backup RAM that can retain stored information even when the ignition key switch is OFF. Based on the stored information in the backup RAM, the CPU determines whether the gas sensor 30 is normal in step 820 of this routine.

If the gas sensor 30 is normal, the CPU determines Yes in step 820, and the processing proceeds to step 830. Then, the CPU detects element impedance for element temperature control (internal resistance of the solid electrolyte body 41s) on the basis of the output current Im at the time when the voltage (for example, a high-frequency voltage) is applied between the first electrode 41a and the second electrode 41b (for example, see Japanese Patent Application Publication No. 10-232220 (JP 10-232220 A) and Japanese Application Publication No. 2002-71633 (JP 2002-71633 A)).

Thereafter, after the CPU sequentially executes the processing in step 840 and step 850, which will be described below, the processing proceeds to step 860. Step 840: the CPU executes heater energization control by target impedance feedback. That is, the CPU controls the energization of the heater 71 such that the element impedance, which is obtained as temperature information in step 830, matches target impedance set in advance (for example, see JP 2002-71633 A and Japanese Patent Application Publication No. 2009-53108 (JP 2009-53108 A)). Step 850: the CPU applies the applied voltage (more specifically, 0.4 V) for the oxygen concentration detection (that is, for A/F detection) between the first electrode 41a and the second electrode 41b. That is, the CPU sets the applied voltage Vm to the applied voltage for the detection of the oxygen concentration.

When the processing proceeds to step 860, the CPU determines whether the gas sensor 30 is activated (whether the sensor is activated). More specifically, the CPU determines whether the temperature of the solid electrolyte body 41s, which is estimated on the basis of the element impedance obtained in step 830, is equal to or higher than an activation temperature threshold. If the gas sensor 30 is not activated, the CPU determines “No” in step 860. Then, the processing proceeds to step 895, and this routine is terminated once.

On the other hand, if the gas sensor 30 is activated, the CPU determines Yes in step 860. Then, the processing proceeds to step 870, and the CPU sets the value of the A/F detection request flag Xaf to “1”. Thereafter, the processing proceeds to step 895, and this routine is terminated once.

Note that, if either one of the value of the A/F detection request flag Xaf and the value of the SOx detection request flag Xs is not “0” at the time point at which the CPU executes the processing in step 810, the CPU determines No in step 810. Then, the processing proceeds to step 895, and this routine is terminated once. In addition, if the gas sensor 30 is not normal at the time point at which the CPU executes the processing in step 820, the CPU determines No in step 820. Then, the processing proceeds to step 895, and this routine is terminated once.

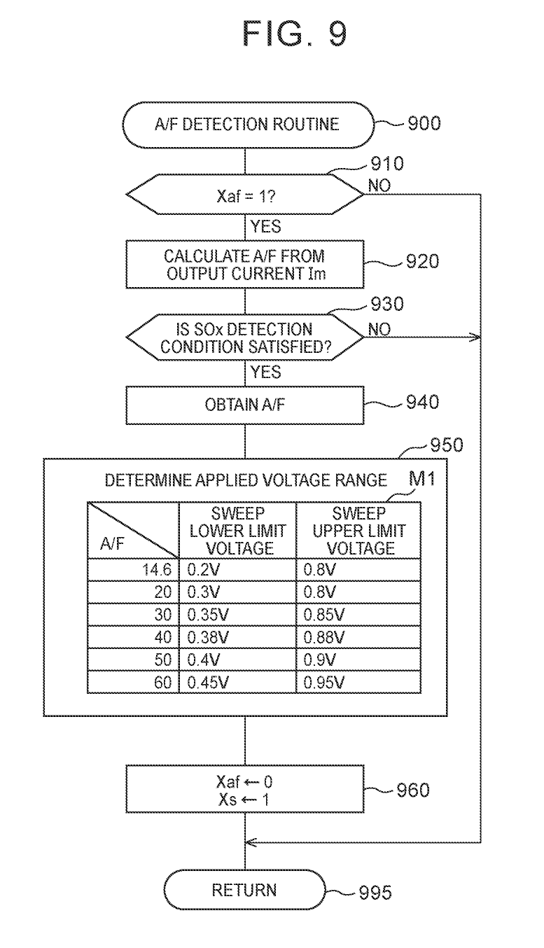

Next, a description will be made on the A/F detection routine with reference to FIG. 9. At predetermined timing, the CPU starts processing from step 900 in FIG. 9. Then, the processing proceeds to step 910, and the CPU determines whether the value of the A/F detection request flag Xaf is “1”.

The A/F detection routine substantially functions in the case where the SOx detection request flag Xs is OFF (Xs=0) after the time point, at which the gas sensor 30 is activated and the value of the A/F detection request flag Xaf is set to “1”, onward. Accordingly, if the value of the A/F detection request flag Xaf is not “1” (that is, if the value of the A/F detection request flag Xaf is “0”), the CPU determines No in step 910. Then, the processing proceeds to step 995, and this routine is terminated once.

On the other hand, if the value of the A/F detection request flag Xaf is set to “1” by the processing in step 870 in FIG. 8, the CPU determines Yes in step 910, and the processing proceeds to step 920. Then, the CPU detects the oxygen concentration on the basis of the output current Im, which is obtained from the gas sensor 30, and applies the oxygen concentration to a predetermined lookup table (also referred to as a “map”) to calculate the engine air-fuel ratio A/F. Note that, in the case where the applied voltage Vm is not set to the applied voltage for the detection of the oxygen concentration (the A/F detection) at a time point at which the processing in step 920 is executed, the CPU sets the applied voltage Vm to the applied voltage for the detection of the oxygen concentration. Thereafter, the processing proceeds to step 930, and the CPU determines whether all conditions that constitute the following SOx detection condition are satisfied on the basis of information obtained from the various sensors (the NE sensor 21, the coolant temperature sensor 22, and the like). The SOx detection condition is established when all of the following conditions are satisfied.

SOx Detection Condition

The internal combustion engine 10 is in a state after being warmed (that is, the coolant temperature THW is equal to or higher than a warming coolant temperature THWth).

The gas sensor 30 is activated.

The internal combustion engine 10 is not in the fuel cut state.

The engine air-fuel ratio A/F is stable. That is, the operation state of the internal combustion engine 10 is an idling state, or a driving state of the vehicle is a steady traveling state. Note that whether the operation state of the internal combustion engine 10 is the idling state is determined by determining whether states where the accelerator pedal operation amount AP is “0” and the engine speed NE is equal to or higher than a predetermined speed continue for a predetermined idling time or longer. Whether the driving state of the vehicle is the steady traveling state is determined by determining whether states where a change amount of the accelerator pedal operation amount AP per unit time is equal to or smaller than a threshold operation change amount and a change amount of a vehicle speed, which is detected by an unillustrated vehicle speed sensor, per unit time is equal to or smaller than a threshold vehicle speed change amount continue for a predetermined steady traveling threshold time or longer. Note that, as the condition that constitutes the SOx detection condition, the following condition may be added.

The SOx concentration is never detected before the ignition key switch is switched to the OFF position after being switched from the OFF position to the ON position (that is, after the start of the internal combustion engine 10 of this time).

If the SOx detection condition is established, the CPU determines Yes in step 930 and sequentially executes processing in step 940 to step 960, which will be described below. Thereafter, the processing proceeds to step 995, and this routine is terminated once.

In step 940, the CPU obtains the A/F that is calculated in step 920. Note that the CPU stores the output current Im, which is used for the calculation of this A/F, in the RAM. In step 950, the CPU determines the voltage range of the applied voltage sweep (the lower limit voltage (the first voltage V1) and the upper limit voltage (the second voltage V2)) by applying the obtained A/F to a lookup table M1. In step 960, the CPU sets the value of the A/F detection request flag Xaf to “0” and sets the value of the SOx detection request flag Xs to “1”.

On the other hand, if at least one of the conditions that constitute the SOx detection condition is not satisfied, the CPU determines No in step 930. Then, the processing proceeds to step 995, and this routine is terminated once.

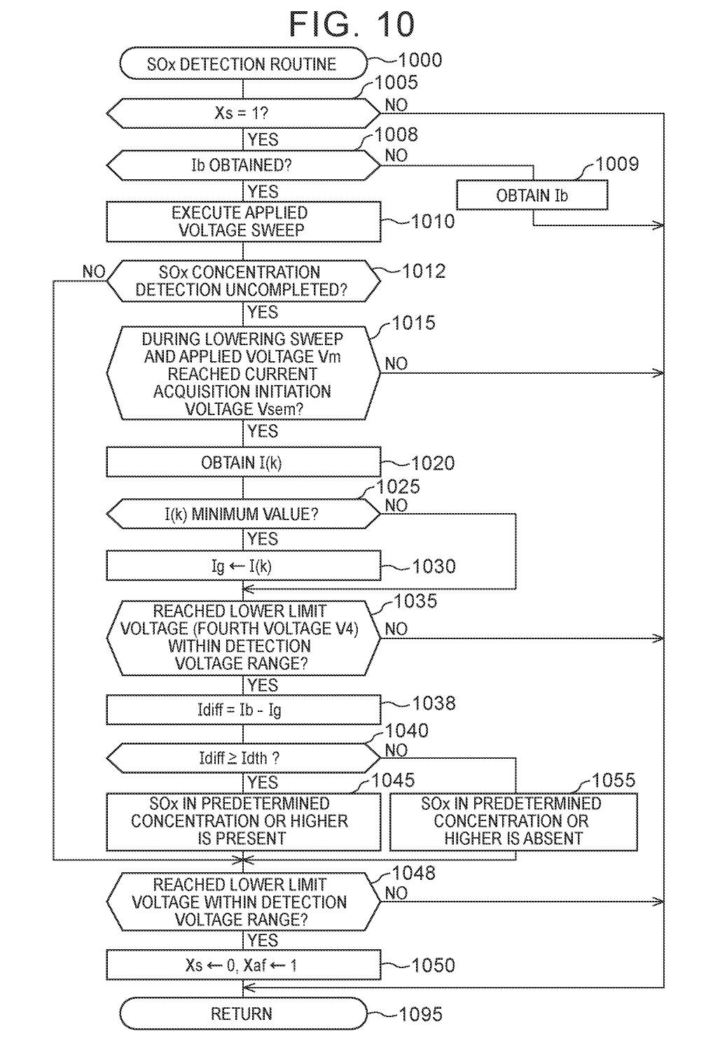

A description will hereinafter be made on the SOx detection routine with reference to FIG. 10. The CPU executes the SOx detection routine, which is shown in the flowchart of FIG. 10, every time predetermined time Δt (2 ms in this example) elapses. At predetermined timing, the CPU starts processing from step 1000 in FIG. 10. Then, the processing proceeds to step 1005, and the CPU determines whether the value of the SOx detection request flag Xs is “1”.

The SOx detection routine substantially functions in the case where the above-described SOx detection condition is established (that is, in the case where the SOx detection request flag Xs is ON (Xs=1)). Accordingly, if the value of the SOx detection request flag Xs is not “1” (that is, the value of the SOx detection request flag Xs is “0”), the CPU determines No in step 1005. Then, the processing proceeds to step 1095, and this routine is terminated once.

On the other hand, if the value of the SOx detection request flag Xs is set to “1” by the processing in step 960 of FIG. 9, the CPU determines Yes in step 1005, and the processing proceeds to step 1008. Then, the CPU determines whether the second current Ib is obtained.

If the second current Ib is not obtained, the CPU determines No in step 1008, and the processing proceeds to step 1009. Then, the CPU obtains the output current Im at the time when the applied voltage Vm, which is stored in the RAM in step 940, is set to the applied voltage for the detection of the oxygen concentration (the A/F detection) as the second current Ib. Thereafter, the processing proceeds to step 1095, and this routine is terminated once.

On the other hand, if the second current Ib is obtained, the CPU determines Yes in step 1008, and the processing proceeds to step 1010. Then, the CPU starts the applied voltage sweep (more specifically, processing to apply the voltage in the sine waveform (a frequency of 1 Hz, for one cycle) at the predetermined sweeping rate within the applied voltage range determined in step 950. In this applied voltage sweep, the boosting sweep is executed first, and the lowering sweep is then executed. If the applied voltage sweep is already being executed at a time point of the processing in step 1010, the CPU continues executing the applied voltage sweep. Note that, if the applied voltage for the detection of the oxygen concentration (the A/F detection), which is set in step 850, is higher than the lower limit value (the first voltage V1) of the applied voltage range determined in step 950, the boosting sweep may be initiated from the applied voltage for the detection of the oxygen concentration (that is, a predetermined voltage that is equal to or higher than the first voltage V1, at which the output current Im is the limiting current for oxygen, and which is lower than the decomposition initiation voltage of sulfur oxides, and that is lower than the decomposition initiation voltage of sulfur oxides).

Thereafter, the processing proceeds to step 1012, and the CPU determines whether the detection of the SOX concentration is uncompleted. If the detection of the SOX concentration is uncompleted, the CPU determines Yes in step 1012. Then, the processing proceeds to step 1015, and the CPU determines whether the current time point is a time point during the lowering sweep and whether the applied voltage Vm has reached the current obtainment initiation voltage Vsem (the third voltage V3). If the determination condition in this step 1015 is not established, the CPU determines No in step 1015. Then, the processing directly proceeds to step 1095, and this routine is terminated once.

On the other hand, if the determination condition in step 1015 is established, the CPU determines Yes in step 1015. Then, the processing proceeds to step 1020, and the CPU obtains the output current Im (=I(k)) at the current time point. Thereafter, the processing proceeds to step 1025, and the CPU determines whether the output current I(k), which is obtained in step 1020, has a minimum value of the output currents I(k) that are obtained by the processing in step 1020 at the time when this routine is executed after the initiation of the currently-executed applied voltage sweep and by the processing in step 1020 at the time when this routine is executed last time. That is, the CPU determines whether I(k)<the first current Ig.

If the output current I(k), which is obtained in step 1020 of this routine, has the minimum value, the CPU determines Yes in step 1025. Then, the processing proceeds to step 1030. After the CPU updates the first current Ig to the output current I(k), the processing proceeds to step 1035. If the output current I(k), which is obtained in step 1020 of this routine, does not have the minimum value, the CPU determines No in step 1025. Then, the processing directly proceeds to step 1035.

In step 1035, the CPU determines whether the applied voltage Vm has reached the lower limit voltage (the fourth voltage V4) within the above-described detection voltage range.

If the applied voltage Vm has not reached the lower limit voltage (the fourth voltage V4) within the detection voltage range, the CPU determines No in step 1035. Then, the processing proceeds to step 1095, and this routine is terminated once.

On the other hand, if the applied voltage Vm has reached the lower limit voltage (the fourth voltage V4) within the detection voltage range, the CPU determines Yes in step 1035, and the processing proceeds to step 1038. Then, the CPU calculates the difference Idiff (=Ib−Ig) that is obtained by subtracting the first current Ig from the second current Ib. Because the difference Idiff is a value that is equal to or larger than 0, the “difference Idiff” is equal to a “magnitude of the difference Idiff”.

Thereafter, the processing proceeds to step 1040, and the CPU determines whether the magnitude of the difference Idiff is equal to or larger than a threshold difference Idth. The threshold difference Idth is a value of the difference Idiff that is appropriate for determining whether SOx in the predetermined concentration or higher is contained in the exhaust gas, and is identified in advance by an experiment or the like. That is, sulfur (S) in an upper limit concentration within a permissible range is mixed in the fuel, and the difference Idiff at the time when the applied voltage sweep is executed under the same condition as above (the same condition as that in the case where the SOx concentration in the exhaust gas is actually detected) is set as the threshold difference Idth. Note that the same condition in this case is that the voltage waveform of the applied voltage sweep, the applied voltage range of the applied voltage sweep, the sweeping rate of the applied voltage sweep, and the like are the same.

If the magnitude of the difference Idiff is equal to or larger than the threshold difference Idth, the reoxidation current change is significant. Accordingly, the CPU determines Yes in step 1040, and the processing proceeds to step 1045. Then, the CPU determines that SOx in the predetermined concentration or higher is contained in the exhaust gas. At this time, the CPU may store that SOx in the predetermined concentration or higher is contained in the exhaust gas (or S exceeding a permissible value is mixed in the fuel) in the backup RAM, and may turn on a predetermined warning lamp.

Next, the processing proceeds to step 1048, and the CPU determines whether the applied voltage Vm has reached the lower limit voltage (the first voltage V1) within the voltage range of the applied voltage sweep. If the applied voltage Vm has not reached the lower limit voltage within the voltage range of the applied voltage sweep, the CPU determines No in step 1048. Then, the processing proceeds to step 1095, and this routine is terminated once. Note that, if the SOx detection routine is executed immediately thereafter, the detection of the SOX concentration is completed (the detection of the SOx concentration is not incomplete). Thus, the CPU determines No in step 1012. Then, the processing proceeds to step 1048, and the CPU executes the processing in step 1048.

If the applied voltage Vm has reached the lower limit voltage within the applied voltage range at the time when the processing in step 1048 is executed, the CPU determines Yes in step 1048. Then, the processing proceeds to step 1050, and the CPU sets the value of the SOx detection request flag Xs to “0” and sets the value of the A/F detection request flag Xaf to “1”. Thereafter, the processing proceeds to step 1095, and this routine is terminated once.

On the other hand, if the magnitude of the difference Idiff is not equal to or higher than the threshold difference Idth, the CPU determines No in step 1040. Then, the processing proceeds to step 1055, and the CPU determines that SOx in the predetermined concentration or higher is not contained in the exhaust gas. At this time, the CPU may store that SOx in the predetermined concentration or higher is not contained in the exhaust gas (or S exceeding the permissible value is not mixed in the fuel) in the backup RAM, and may turn off the predetermined warning lamp. After the processing proceeds to step 1048, in accordance with the determination result of step 1048, the processing directly proceeds to step 1095, and this routine is terminated once. Alternatively, the processing proceeds to step 1095 via step 1050, and this routine is terminated once.

As it has been described so far, the ECU 20 of the first detector calculates the difference Idiff (=Ib−Ig), which is obtained by subtracting the first current Ig from the second current Ib, as the parameter representing the degree of the reoxidation current change of sulfur that is less likely to be influenced by the oxygen containing components other than SOx contained in the exhaust gas and that is less likely to be influenced by the concentration of oxygen contained in the exhaust gas during the measurement. Furthermore, the ECU 20 determines whether SOx in the predetermined concentration or higher is contained in the exhaust gas on the basis of the calculated difference Idiff. At the time, the ECU 20 appropriately sets the sweeping rate of the lowering sweep, the voltage range of the applied voltage sweep, and the like such that the large degree of the reoxidation current change appears. Then, the ECU 20 obtains the difference Idiff.

More specifically, if the difference Idiff (the magnitude of the difference Idiff) is equal to or larger than the threshold difference Idth, the ECU 20 determines that SOx in the predetermined concentration or higher is contained in the exhaust gas. If the above difference Idiff (the magnitude of the difference Idiff) is smaller than the threshold difference Idth, the ECU 20 determines that SOx in the predetermined concentration or higher is not contained in the exhaust gas. Accordingly, the ECU 20 can accurately determine the presence or the absence of SOx in the predetermined concentration or higher contained in the exhaust gas.

### Second Embodiment

Next, a description will be made on a gas detector according to a second embodiment of the disclosure (hereinafter may also be referred to as a “second detector”). The second detector differs from the first detector in a point that, instead of the output current Im at the time before the applied voltage sweep for the detection of the SOx concentration is initiated and when the applied voltage Vm is set to the applied voltage for the detection of the oxygen concentration, the output current Im at the time when the applied voltage Vm becomes the upper limit voltage (the second voltage V2) of the applied voltage sweep is used as the second current Ib.

Overview of Actuation

The second detector detects the reoxidation current change by using a parameter (the difference Idiff), which will be described below, and thereby detects the SOx concentration (actually, determines the presence or the absence of SOx in the predetermined concentration or higher).

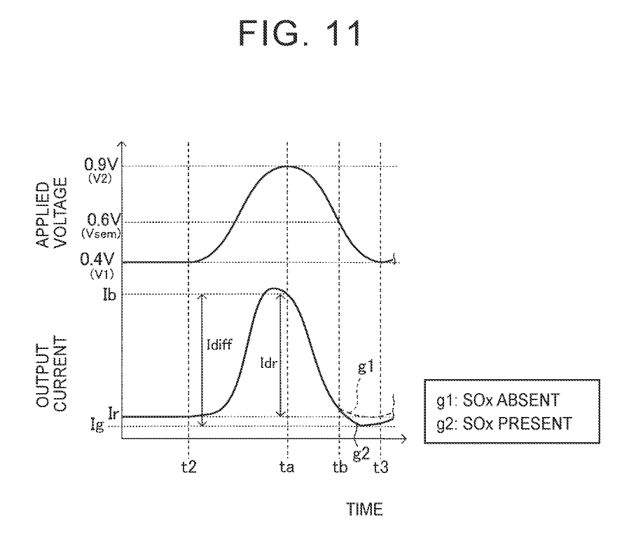

More specifically, as shown in FIG. 11, the second detector obtains the output current Im at the time (time ta) when the applied voltage Vm is the upper limit voltage (the second voltage V2) of the applied voltage sweep as the second current Ib. This second current Ib is theoretically changed under the influence of the SOx concentration in the exhaust gas. However, because the SOx concentration in the exhaust gas is extremely low, it is considered that the second current Ib does not depend on the SOx concentration in the exhaust gas. Furthermore, the second detector obtains the minimum value of the output current Im (the output current Im indicated by a line g2) in a period (the period from the time tb to the time t3) in which the applied voltage Vm is within a range (that is, the detection voltage range) between not less than the fourth voltage V4, which is higher than the first voltage V1, and not more than the current obtainment initiation voltage Vsem (0.6 V) during the lowering sweep, and obtains this minimum value of the output current Im as the first current Ig. Moreover, the second detector calculates the difference Idiff (=Ib−Ig) that is obtained by subtracting the first current Ig from the second current Ib. The second detector detects the SOx concentration (actually, determines the presence or the absence of SOx in the predetermined concentration or higher) on the basis of the difference Idiff.

Specific Actuation

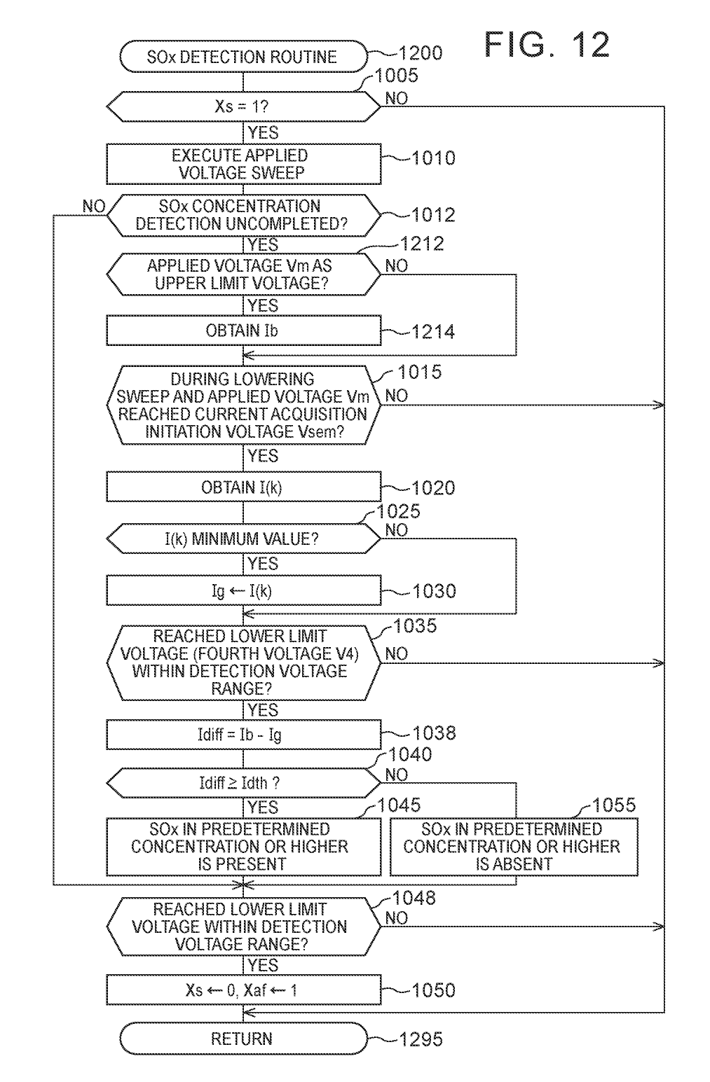

Next, a description will be made on specific actuation of the second detector. Every time predetermined time elapses, the CPU of the ECU 20 executes the same sensor activation determination routine as the routine of FIG. 8, the same A/F detection routine as the routine of FIG. 9, and a SOx detection routine shown in FIG. 12.

The sensor activation determination routine and the A/F detection routine are respectively the same as those routines executed by the first detector and have already been described. Thus, the description thereon will not be made.

A description will hereinafter be made on the SOx detection routine with reference to FIG. 12. The routine in FIG. 12 differs from the routine in FIG. 10 only in points that step 1008 and step 1009 of the routine in FIG. 10 are deleted and that step 1212 and step 1214 are added between step 1012 and step 1015. Accordingly, a description will hereinafter be centered on these different points.

If the value of the SOx detection request flag Xs is set to “1”, the processing proceeds from step 1005 to step 1010. Then, after executing the processing in step 1010 (that is, the applied voltage sweep), the CPU determines whether the detection of the SOx concentration is uncompleted in step 1012.

If the detection of the SOx concentration is uncompleted, the processing proceeds to step 1212, and the CPU determines whether the applied voltage Vm matches the upper limit voltage (the second voltage V2).

If the applied voltage Vm matches the upper limit voltage, the CPU determines Yes in step 1212, and the processing proceeds to step 1214. Then, after the CPU obtains the output current Im at the time when the applied voltage Vm is the upper limit voltage as the second current Ib, the processing proceeds to step 1015. On the other hand, if the applied voltage Vm is not the upper limit voltage, the CPU determines No in step 1212, and the processing proceeds to step 1015.

Thereafter, the CPU sequentially executes processing in an appropriate step(s) from step 1015 to step 1055. Then, the processing proceeds to step 1295, and this routine is terminated once.

As it has been described so far, the second detector obtains the output current Im at the time point at which the applied voltage Vm becomes the second voltage V2 during the applied voltage sweep as the second current Ib, and obtains the minimum value of the output current Im in the period in which the applied voltage Vm is the voltage within the detection voltage range during the following lowering sweep as the first current Ig. Accordingly, because a period from the time point at which the second current Ib is obtained to the time point at which the first current Ig is obtained can be shortened, there is a low possibility that the oxygen concentration in the exhaust gas during the period is significantly changed. Thus, degrees of the influence of the oxygen concentration in the exhaust gas on the second current Ib and the first current Ig can substantially match each other. As a result, because the difference Idiff (=Ib−Ig) becomes a value that is less likely to be influenced by the oxygen concentration in the exhaust gas and that accurately corresponds to the SOx concentration in the exhaust gas, the detection of the SOx concentration (actually, the determination on the presence or the absence of SOx in the predetermined concentration or higher) can further accurately be made.

### Third Embodiment

Next, a description will be made on a gas detector according to a third embodiment of the disclosure (hereinafter may also be referred to as a “third detector”). The third detector differs from the first detector in a point that, instead of the output current Im at the time before the applied voltage sweep for the detection of the SOx concentration is initiated and when the applied voltage Vm is set as the applied voltage for the detection of the oxygen concentration, the output current Im at the time when the applied voltage Vm becomes the lower limit voltage (the first voltage V1) of the applied voltage sweep is used as the second current Ib.

Overview of Actuation

The third detector detects the reoxidation current change by using a parameter (the difference Idiff), which will be described below, and thereby detects the SOx concentration (actually, determines the presence or the absence of SOx in the predetermined concentration or higher).

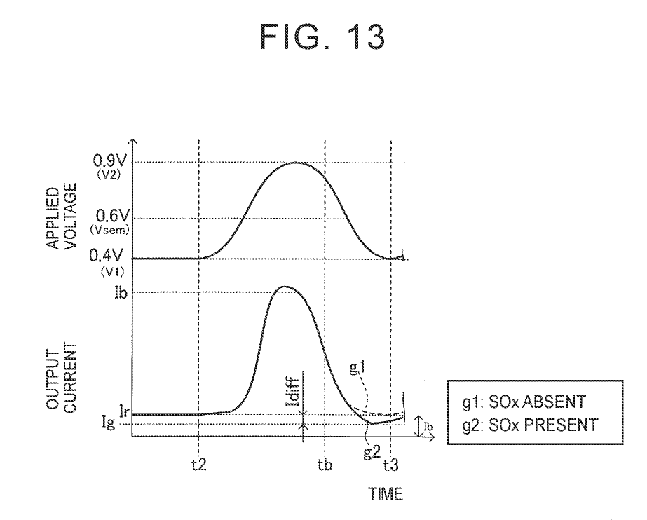

More specifically, as shown in FIG. 13, similar to the first and second detectors, the third detector obtains the minimum value of the output current Im (the output current Im indicated by a line g2) in the period (the period from the time tb to the time t3) in which the applied voltage Vm is equal to or lower than the current obtainment initiation voltage Vsem (0.6 V) during the lowering sweep, and obtains the minimum value of the output current Im as the first current Ig. Furthermore, the third detector obtains the output current Im at the time (the time t3) when the applied voltage Vm is the lower limit voltage (the first voltage V1) of the applied voltage sweep as the second current Ib. This second current Ib is the output current Im that is obtained at the time when the applied voltage Vm is lower than the SOx decomposition initiation voltage and the reoxidation reaction of S that has been adsorbed to the first electrode 41a (the negative electrode) is substantially terminated by the lowering sweep. In addition, the third detector calculates the difference Idiff (=Ib−Ig) that is obtained by subtracting the first current Ig from the second current Ib. Moreover, the third detector detects the SOx concentration (actually, determines the presence or the absence of SOx in the predetermined concentration or higher) on the basis of the difference Idiff.

Note that the third detector may maintain the applied voltage Vm at the first voltage V1 for a predetermined time after the applied voltage Vm reaches the lower limit voltage (the first voltage V1) of the applied voltage sweep during the applied voltage sweep, and may obtain the output current Im, which is obtained in a period of this predetermined time, as the second current Ib. In this case, because the output current Im obtained in the period is not changed by the SOx concentration in the exhaust gas, either, the output current Im can be used as the second current Ib.

Specific Actuation

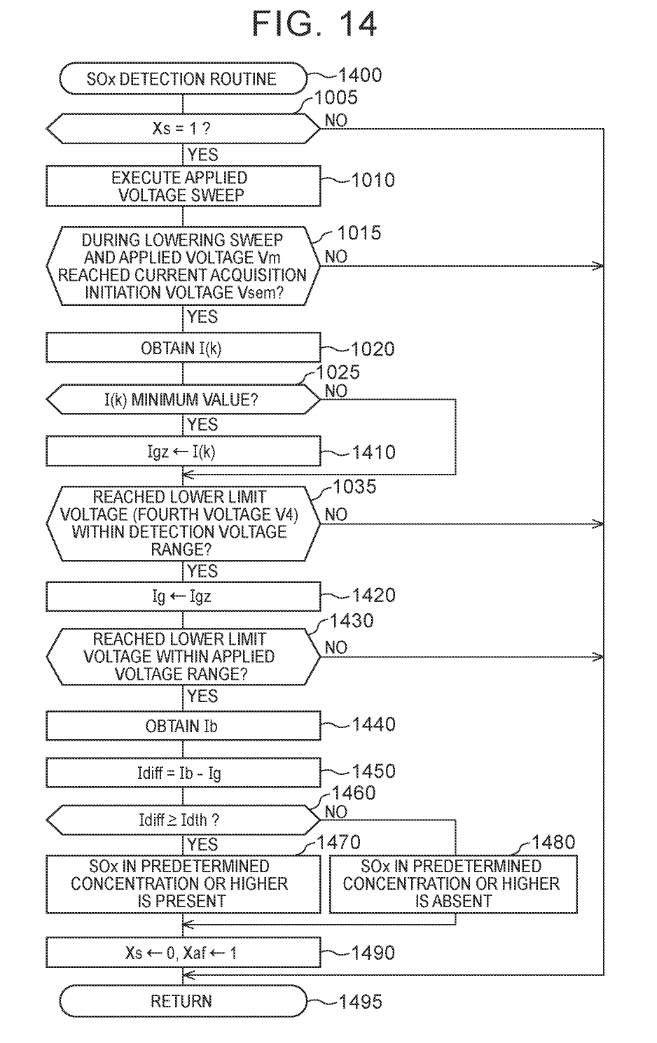

Next, a description will be made on specific actuation of the third detector. Every time predetermined time elapses, the CPU of the ECU 20 executes the same sensor activation determination routine as the routine of FIG. 8, the same A/F detection routine as the routine of FIG. 9, and a SOx detection routine shown in FIG. 14.

The sensor activation determination routine and the A/F detection routine are respectively the same as those routines executed by the first detector and have already been described. Thus, the description thereon will not be made.

A description will hereinafter be made on the SOx detection routine with reference to FIG. 14. The routine of FIG. 14 differs from the routine of FIG. 10 only in the following points.
Step 1008, step 1009, and step 1012 of FIG. 10 are deleted.
Step 1030 of FIG. 10 is replaced with step 1410.
Step 1038 to step 1055 of FIG. 10 are replaced with step 1420 to step 1490. Hereinafter, the description will be centered on these different points.

In step 1410, The CPU updates a temporal value Igz of the first current to the output current 1(k), and then the processing proceeds to step 1035. If the output current I(k) obtained in step 1020 of this routine does not have a minimum value, the CPU determines No in step 1025, and the processing directly proceeds to step 1035.

If the applied voltage Vm has reached the lower limit voltage (the fourth voltage V4) within the above-described detection voltage range, the CPU determines Yes in step 1035, and the processing proceeds to step 1420. Then, the CPU stores the temporal value Igz of the first current as the first current Ig. Next, the processing proceeds to step 1430, and the CPU determines whether the applied voltage Vm has reached the lower limit voltage (the first voltage V1) of the applied voltage sweep. If the applied voltage Vm has not reached the lower limit voltage (the first voltage V1) of the applied voltage sweep, the processing proceeds to step 1495, and this routine is terminated once.

On the other hand, if the applied voltage Vm has reached the lower limit voltage (the first voltage V1) of the applied voltage sweep, the CPU determines Yes in step 1430 and sequentially executes processing in step 1440 and step 1450, which will be described below. Then, the processing proceeds to step 1460. In step 1440, the CPU obtains the output current Im at the time when the applied voltage Vm is the lower limit voltage (the first voltage V1) as the second current Ib. In step 1450, the CPU calculates the difference Idiff (=Ib−Ig) that is obtained by subtracting the first current Ig from the second current Ib. Because the difference Idiff is the value that is equal to or larger than 0, the “difference Idiff” is equal to the “magnitude of the difference Idiff”.

Thereafter, the processing proceeds to step 1460, and the CPU determines whether the magnitude of the difference Idiff is equal to or larger than the threshold difference Idth. The threshold difference Idth is an appropriate value of the difference Idiff that is used to determine whether SOx in the predetermined concentration or higher is contained in the exhaust gas, and is identified by the experiment or the like in advance.

If the magnitude of the difference Idiff is equal to or larger than the threshold difference Idth, the reoxidation current change is significant. Accordingly, the CPU determines Yes in step 1460, and the processing proceeds to step 1470. Then, the CPU determines that SOx in the predetermined concentration or higher is contained in the exhaust gas. At this time, the CPU may store that SOx in the predetermined concentration or higher is contained in the exhaust gas (or S exceeding the permissible value is mixed in the fuel) in the backup RAM, and may turn on the predetermined warning lamp. Thereafter, the processing proceeds to step 1490, and the CPU sets the value of the SOx detection request flag Xs to “0” and sets the value of the A/F detection request flag Xaf to “1”. Then, the processing proceeds to step 1495, and this routine is terminated once.

On the other hand, if the magnitude of the difference Idiff is not equal to or larger than the threshold difference Idth, the CPU determines No in step 1460. Then, the processing proceeds to step 1480, and the CPU determines that SOx in the predetermined concentration or higher is not contained in the exhaust gas. At this time, the CPU may store that SOx in the predetermined concentration or higher is not contained in the exhaust gas (or S exceeding the permissible value is not mixed in the fuel) in the backup RAM, and may turn off the predetermined warning lamp. Thereafter, the CPU executes the processing in step 1490. Then, the processing proceeds to step 1495, and this routine is terminated once.

As it has been described so far, the third detector obtains the minimum value of the output current Im in the period in which the applied voltage Vm is the voltage that is within the detection voltage range during the lowering sweep as the first current Ig, and thereafter obtains the output current Im at the time point at which the applied voltage Vm becomes the first voltage V1 during the lowering sweep as the second current Ib. Accordingly, because a period from the time point at which the first current Ig is obtained to the time point at which the second current Ib is obtained can be shortened, there is the low possibility that the oxygen concentration in the exhaust gas during the period is significantly changed. Thus, the degrees of the influence of the oxygen concentration in the exhaust gas on the first current Ig and the second current Ib can substantially match each other. As a result, because the difference Idiff (=Ib−Ig) becomes the value that is less likely to be influenced by the oxygen concentration in the exhaust gas and that accurately corresponds to the SOx concentration in the exhaust gas, the detection of the SOx concentration (actually, the determination on the presence or the absence of SOx in the predetermined concentration or higher) can further accurately be made.

### MODIFIED EXAMPLES

The specific description has been made so far on each of the embodiments. However, the disclosure is not limited to each of the above-described embodiments, and various modified examples that are based on the technical idea of the disclosure can be adopted.

In each of the above-described embodiments, the first current Ig is not limited to the minimum value of the output current Im in the period in which the applied voltage Vm is within the detection voltage range during the above-described lowering sweep. As long as the first current Ig has a value that is correlated with the output current Im in the period in which the applied voltage Vm is within the detection voltage range during the lowering sweep, this first current Ig may be obtained as the first current Ig. For example, in each of the embodiments, the output current Im at the time when the applied voltage Vm is a current obtainment voltage Vg during the lowering sweep may be obtained as the first current Ig. In this case, the current obtainment voltage Vg is selected from a range (the detection voltage range) between the fourth voltage V4 and the third voltage Vsem inclusive. The fourth voltage V4 is higher than the lower limit voltage (the first voltage V1). The third voltage Vsem is equal to or lower than the SOx decomposition initiation voltage (0.6 V).

The output current Im that can be used as the second current Ib is not limited to the output current Im that is obtained as described in each of the embodiments. The output current Im other than these may be used as the second current Ib. More specifically, as long as the output current Im is the output current Im at a time when the applied voltage Vm becomes a voltage at which the magnitude of the output current Im in the case where SOx is contained in the exhaust gas and the magnitude of the output current Im in the case where SOx is not contained in the exhaust gas are the same, when the oxygen concentration in the exhaust gas can be regarded to be equal to the oxygen concentration in the exhaust gas at the time when the first current Ig is obtained, and when a decomposition current of oxygen in the concentration is included in the second current Ib, the output current Im at the time may be used as the second current Ib.

In each of the above-described embodiments, it is determined whether SOx in the predetermined concentration or higher is contained in the exhaust gas by comparing the magnitude of the difference Idiff and the threshold difference Idth. However, as will be described below, the SOx concentration in the exhaust gas may be obtained on the basis of the difference Idiff.

### Modified Example of First Detector

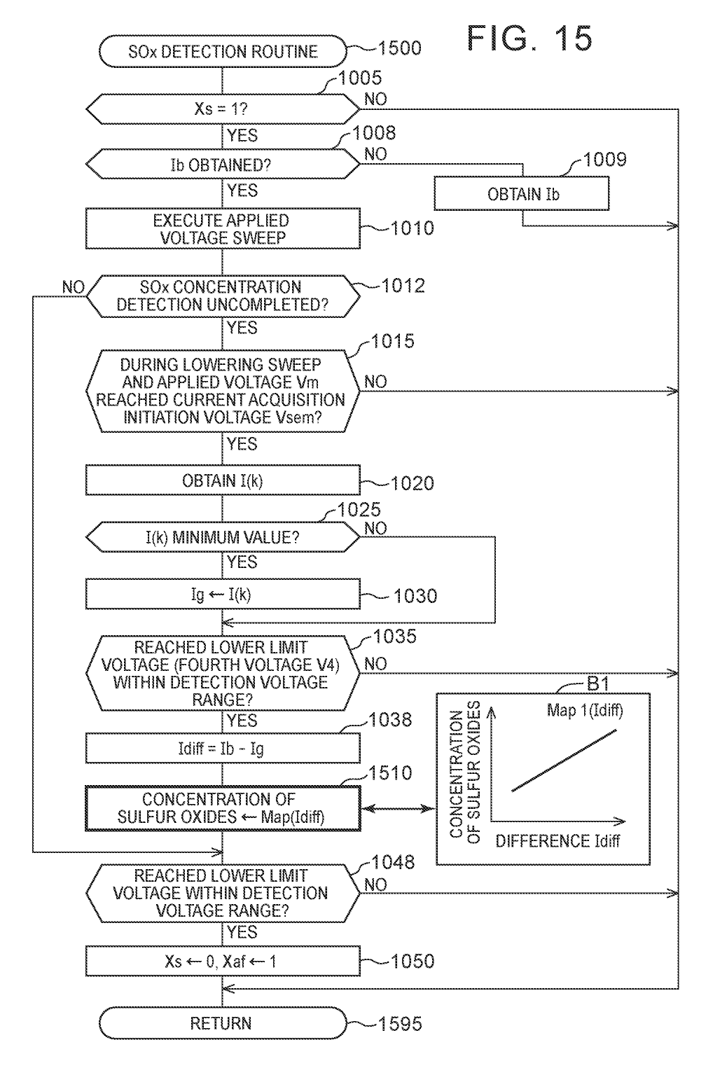

For example, instead of the SOx concentration detection routine shown in FIG. 10, the CPU can be configured to execute a SOx concentration detection routine shown in FIG. 15. This routine shown in FIG. 15 is a routine in which processing in step 1510 is executed instead of the processing in step 1040, step 1045, and step 1055 of the routine shown in FIG. 10. Thus, hereinafter, a description will primarily be made on the processing in step 1510 of FIG. 15.

The CPU obtains the output current Im at the time when the applied voltage Vm is set to the applied voltage for the detection of the oxygen concentration as the second current Ib in step 1009 of FIG. 15, and obtains the minimum value of the output current Im at a time when the applied voltage Vm is within the detection voltage range (V4 to Vsem) as the first current Ig in step 1030 of FIG. 15.

Then, the CPU calculates the difference Idiff (=Ib−Ig) in step 1038 of FIG. 15. The processing then proceeds to step 1510, and the CPU applies the difference Idiff to a lookup table Map 1(Idiff) and thereby obtains the SOx concentration in the exhaust gas. Note that the ROM (the memory section) of the ECU 20 stores a relationship between the difference Idiff and the SOx concentration in the exhaust gas as the lookup table Map 1(Idiff) (see a block B1 in FIG. 15). This lookup table can be obtained by an experiment or the like in advance.

### Modified Example of Second Detector

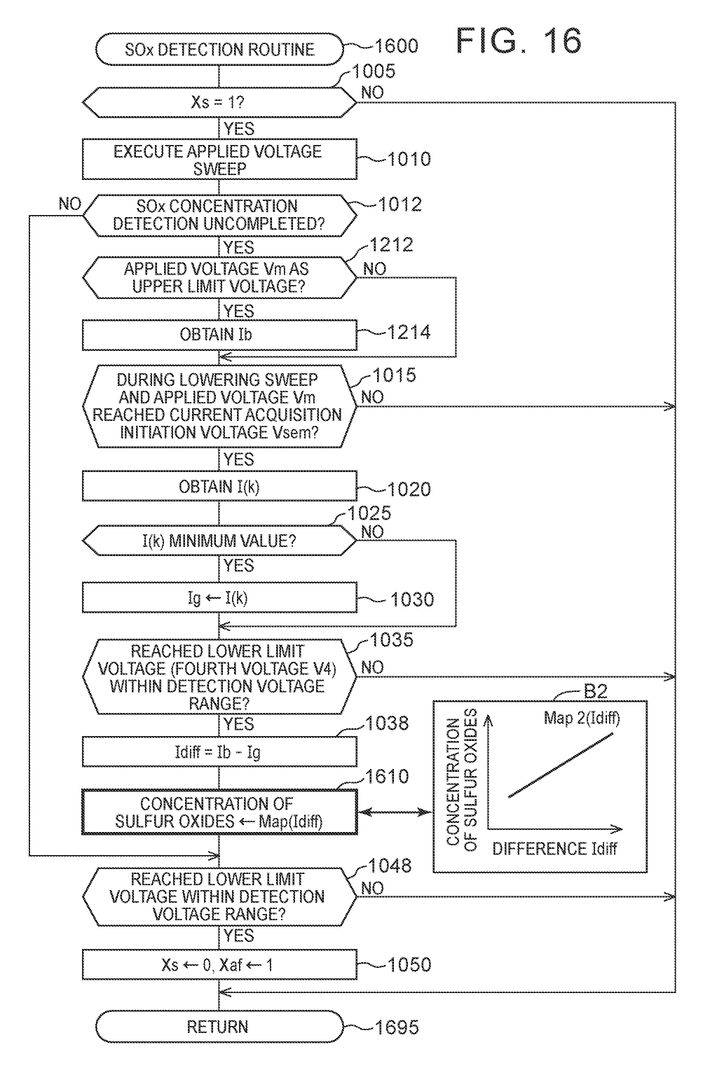

Furthermore, for example, instead of the SOx concentration detection routine shown in FIG. 12, the CPU can be configured to execute a SOx concentration detection routine shown in FIG. 16. This routine shown in FIG. 16 is a routine in which processing in step 1610 is executed instead of the processing in step 1040, step 1045, and step 1055 of the routine shown in FIG. 12. Thus, a description will primarily be made on the processing in step 1610 in FIG. 16.

The CPU obtains the output current Im at the time when the applied voltage Vm is the upper limit voltage (the second voltage V2) of the applied voltage sweep as the second current Ib in step 1214 of FIG. 16, and obtains the minimum value of the output current Im at the time when the applied voltage Vm is within the detection voltage range (V4 to Vsem) as the first current Ig in step 1030 of FIG. 16.

Then, the CPU calculates the difference Idiff (=Ib−Ig) in step 1038 of FIG. 16. The processing then proceeds to step 1610, and the CPU applies the difference Idiff to a lookup table Map 2(Idiff) and thereby obtains the SOx concentration in the exhaust gas. Note that the ROM (the memory section) of the ECU 20 stores a relationship between the difference Idiff and the SOx concentration in the exhaust gas as the lookup table Map 2(Idiff) (see a block B2 in FIG. 16). This lookup table can be obtained by an experiment or the like in advance.

### Modified Example of Third Detector

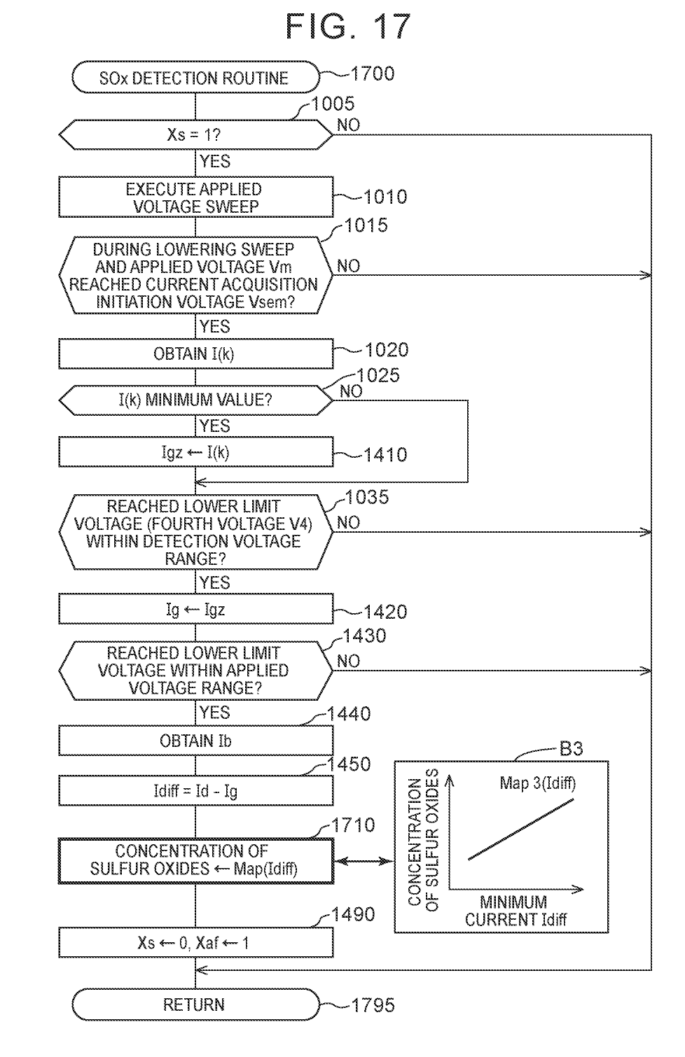

Furthermore, for example, instead of the SOx concentration detection routine shown in FIG. 14, the CPU can be configured to execute a SOx concentration detection routine shown in FIG. 17. This routine shown in FIG. 17 is a routine in which processing in step 1710 is executed instead of the processing in step 1460, step 1470, and step 1480 of the routine shown in FIG. 14. Thus, a description will primarily be made on the processing in step 1710 in FIG. 17.

The CPU obtains the output current Im at the time when the applied voltage Vm is within the detection voltage range (V4 to Vsem) as the first current Ig in step 1420 of FIG. 17, and obtains the output current Im at the time when the applied voltage Vm is the lower limit voltage (the first voltage V1) of the applied voltage sweep as the second current Ib in step 1440 of FIG. 17.

Then, the CPU calculates the difference Idiff (=Ib−Ig) in step 1450 of FIG. 17. The processing then proceeds to step 1710, and the CPU applies the difference Idiff to a lookup table Map 3(Idiff) and thereby obtains the SOx concentration in the exhaust gas. Note that the ROM (the memory section) of the ECU 20 stores a relationship between the difference Idiff and the SOx concentration in the exhaust gas as the lookup table Map 3(Idiff) (see a block B3 in FIG. 17). This lookup table can be obtained by an experiment or the like in advance.

Each of the ECUs 20 in these modified examples is configured to use the difference Idiff as the parameter representing the reoxidation current change that is less likely to be influenced by the oxygen containing components other than SOx contained in the exhaust gas and to obtain the SOx concentration in the exhaust gas that corresponds to the above difference Idiff from the lookup table stored in the ROM. Therefore, the concentration of sulfur oxides in the exhaust gas can accurately be detected.

Furthermore, for example, in each of the above-described embodiments, the engine air-fuel ratio A/F is obtained in step 940 and step 950 of FIG. 9, and the lower limit voltage and the upper limit voltage within the voltage range of the applied voltage sweep are determined on the basis of the obtained A/F. However, each of the above-described embodiments may be configured as follows.

More specifically, in each of the above-described embodiments, the oxygen concentration may be detected on the basis of the output current Im in the case where the applied voltage Vm is set to the applied voltage for the detection of the oxygen concentration in step 920, and the lower limit voltage and the upper limit voltage within the voltage range of the applied voltage sweep may be determined on the basis of the oxygen concentration in step 950. In this case, the lookup table M1 is a table that defines a relationship between the oxygen concentration and a combination of the lower limit voltage and the upper limit voltage within the voltage range of the applied voltage sweep.

Similarly, in each of the above-described embodiments, the output current Im in the case where the applied voltage Vm is set to the applied voltage for the detection of the oxygen concentration may be detected in step 920, and the lower limit voltage and the upper limit voltage within the voltage range of the applied voltage sweep may be determined on the basis of the output current 1m itself in step 950. In this case, the lookup table M1 is a table that defines a relationship between the output current Im and the combination of the lower limit voltage and the upper limit voltage within the voltage range of the applied voltage sweep.

Furthermore, for example, the voltage waveform of the applied voltage sweep in each of the embodiments and each of the modified examples is not limited to the waveforms shown in FIG. 3B and FIG. 3C and may be an arbitrary waveform (for example, a triangular wave) as long as the reoxidation current change, which is resulted from the reoxidation reaction of sulfur that has been adsorbed to the first electrode 41a, becomes extremely significant from a certain time point during the lowering sweep by appropriately setting the lowering rate.

What is claimed is:

1. A gas detector comprising:
an element provided in an exhaust passage of an internal combustion engine, the element including an electrochemical cell and a diffusion resistance body, the electrochemical cell including a solid electrolyte body, a first electrode, and a second electrode, the solid electrolyte body having oxide ion conductivity, the first electrode and the second electrode being respectively provided on surfaces of the solid electrolyte body, the diffusion resistance body being made of a porous material through which exhaust gas flowing through the exhaust passage can pass, and the element being configured that the exhaust gas flowing through the exhaust passage reaches the first electrode through the diffusion resistance body;
a voltage application device configured to apply a voltage between the first electrode and the second electrode;
a current detector configured to detect an output current that is a current flowing between the first electrode and the second electrode; and
an electronic control unit configured to control an applied voltage that is the voltage applied between the first electrode and the second electrode by the voltage application device,
the electronic control unit being configured to either determine whether sulfur oxides in a predetermined concentration or higher are contained in the exhaust gas or detect a concentration of the sulfur oxides in the exhaust gas, based on the output current detected by the current detector,
when an air-fuel ratio of air mixture supplied to the internal combustion engine is in a stable state, the electronic control unit being configured to execute boosting sweep for boosting the applied voltage from a predetermined voltage to a second voltage by the voltage application device and then execute lowering sweep for lowering the applied voltage from the second voltage to a first voltage at a predetermined lowering rate, the predetermined voltage being a voltage that is equal to or higher than the first voltage and is lower than a decomposition initiation voltage of the sulfur oxides, the first voltage being a voltage that is lower than the decomposition initiation voltage of the sulfur oxides, the first voltage being a voltage when the output current becomes a limiting current of oxygen, and the second voltage being a voltage that is higher than the decomposition initiation voltage of the sulfur oxides,
the electronic control unit being configured to obtain a parameter that is correlated with a degree of a change occurred to the output current based on the output current detected by the current detector, the change occurred to the output current being a change in the output current resulted from a current flowing between the first electrode and the second electrode when a reoxidation reaction of sulfur that has been adsorbed to the first electrode leads to generation of the sulfur oxides in the first electrode when the applied voltage becomes lower than the decomposition initiation voltage of the sulfur oxides during the lowering sweep, and the degree of the change occurred to the output current is increased as the concentration of the sulfur oxides contained in the exhaust gas is increased,
the electronic control unit being configured to either determine whether sulfur oxides in the predetermined concentration or higher are contained in the exhaust gas or detect the concentration the of sulfur oxides in the exhaust gas, based on the parameter,
the electronic control unit being configured to set the predetermined lowering rate such that a rate of the reoxidation reaction becomes a rapidly increased rate at a time when the applied voltage becomes a voltage within a range between the first voltage and the decomposition initiation voltage of the sulfur oxides,
the electronic control unit being configured to obtain a value that is correlated with the output current in a predetermined period as a first current based on the output current detected by the current detector, the predetermined period being a period in which the lowering sweep is executed and in which the applied voltage is within a range between the first voltage and the decomposition initiation voltage of the sulfur oxides, the first voltage being excluded from the range, and the decomposition initiation voltage being included in the range,
the electronic control unit being configured to obtain a second current, the second current being the output current detected by the current detector at a time when the applied voltage is a particular voltage that is equal to or higher than a voltage at which the output current is the limiting current of oxygen, and the second current being the output current that exclude a current resulted from the reoxidation reaction of sulfur that has been adsorbed to the first electrode in the first electrode by the boosting sweep, and
the electronic control unit being configured to calculate a difference between the obtained second current and the obtained first current and use the difference as the parameter.

2. The gas detector according to claim 1, wherein
the electronic control unit is configured to determine whether sulfur oxides in the predetermined concentration or higher are contained in the exhaust gas,
the electronic control unit determines whether a magnitude of the difference is equal to or larger than a predetermined threshold,
the electronic control unit is configured to determine that sulfur oxides in the predetermined concentration or higher are contained in the exhaust gas when the electronic control unit determines that the magnitude of the difference is equal to or larger than the predetermined threshold, and
the electronic control unit is configured to determine that sulfur oxides in the predetermined concentration or higher are not contained in the exhaust gas when the electronic control unit determines that the magnitude of the difference is smaller than the predetermined threshold.

3. The gas detector according to claim 1, wherein
the electronic control unit is configured to detect the concentration of the sulfur oxides in the exhaust gas, and
the electronic control unit is configured to detect the concentration of the sulfur oxides in the exhaust gas based on the difference.

4. The gas detector according to claim 1, wherein
the electronic control unit is configured to obtain a minimum value of the output current detected by the current detector in a period in which the lowering sweep is executed and in which the applied voltage is in a detection voltage range as the first current,
the detection voltage range is a range between a fourth voltage and a third voltage inclusive,
the third voltage is a voltage that is equal to or lower than the decomposition initiation voltage of the sulfur oxides, and
the fourth voltage is a voltage that is higher than the first voltage.

5. The gas detector according to claim 1, wherein
the electronic control unit is configured to obtain the output current that is detected by the current detector when the lowering sweep is executed and the applied voltage becomes a current obtainment voltage as the first current,
the current obtainment voltage is a voltage selected from a detection voltage range,
the detection voltage range is a range between a fourth voltage and a third voltage inclusive,
the third voltage is a voltage that is equal to or lower than the decomposition initiation voltage of the sulfur oxides, and
the fourth voltage is a voltage that is higher than the first voltage.

6. The gas detector according to claim 1, wherein
the electronic control unit is configured to adopt an applied voltage for detection of an air-fuel ratio, at which the output current becomes the limiting current of oxygen, as the particular voltage,
the electronic control unit is configured to set the applied voltage to the applied voltage for the detection of the air-fuel ratio by the voltage application device before starting execution of the boosting sweep, and
the electronic control unit is configured to obtain the output current that is detected by the current detector when the applied voltage is set as the applied voltage for the detection of the air-fuel ratio as the second current.

7. The gas detector according to claim 1, wherein
the electronic control unit is configured to obtain the output current that is detected by the current detector when the applied voltage becomes the second voltage during the boosting sweep as the second current.

8. The gas detector according to claim 1, wherein
the electronic control unit is configured to obtain the output current that is detected by the current detector when the applied voltage becomes the first voltage during the lowering sweep as the second current.

9. A control method for a gas detector,
the gas detector including an element, a voltage application device, a current detector, and an electronic control unit,
the element being provided in an exhaust passage of an internal combustion engine, the element including an electrochemical cell and a diffusion resistance body, the electrochemical cell including a solid electrolyte body, a first electrode, and a second electrode, the solid electrolyte body having oxide ion conductivity, the first electrode and the second electrode being respectively provided on surfaces of the solid electrolyte body, the diffusion resistance body being made of a porous material through which exhaust gas flowing through the exhaust passage can pass, and the element being configured that the exhaust gas flowing through the exhaust passage reaches the first electrode through the diffusion resistance body,
the voltage application device being configured to apply a voltage between the first electrode and the second electrode, and
the current detector being configured to detect an output current that is a current flowing between the first electrode and the second electrode,
the control method comprising:
controlling an applied voltage that is the voltage applied between the first electrode and the second electrode by the voltage application device;
either determining, by the electronic control unit, whether sulfur oxides in a predetermined concentration or higher are contained in the exhaust gas or detecting, by the electronic control unit, a concentration of the sulfur oxides in the exhaust gas, based on the output current detected by the current detector;
when an air-fuel ratio of air mixture that is supplied to the internal combustion engine is in a stable state, executing, by the electronic control unit, boosting sweep for boosting the applied voltage from a predetermined voltage to a second voltage by the voltage application device and then executing, by the electronic control unit, lowering sweep for lowering the applied voltage from the second voltage to a first voltage at a predetermined lowering rate, the predetermined voltage being a voltage that is equal to or higher than the first voltage and is lower than a decomposition initiation voltage of the sulfur oxides, the first voltage being a voltage that is lower than the decomposition initiation voltage of the sulfur oxides, the first voltage being a voltage when the output current becomes a limiting current of oxygen, and the second voltage being a voltage that is higher than the decomposition initiation voltage of sulfur oxides;
obtaining, by the electronic control unit, a parameter that is correlated with a degree of a change occurred to the output current based on the output current detected by the current detector, the change occurred to the output current being a change in the output current resulted from a current flowing between the first electrode and the second electrode when a reoxidation reaction of sulfur that has been adsorbed to the first electrode leads to generation of the sulfur oxides in the first electrode when the applied voltage becomes lower than the decomposition initiation voltage of sulfur oxides during the lowering sweep, and the degree of the change occurred to the output current being increased as the concentration of the sulfur oxides contained in the exhaust gas is increased;
either determining, by the electronic control unit, whether sulfur oxides in the predetermined concentration or higher are contained in the exhaust gas or detecting, by the electronic control unit, the concentration of the sulfur oxides in the exhaust gas, based on the parameter;
setting, by the electronic control unit, the predetermined lowering rate such that a rate of the reoxidation reaction becomes a rapidly increased rate at a time when the applied voltage becomes a voltage within a range between the first voltage and the decomposition initiation voltage of the sulfur oxides;
obtaining, by the electronic control unit, a value that is correlated with the output current in a predetermined period as a first current based on the output current detected by the current detector, the predetermined period being a period in which the lowering sweep is executed and in which the applied voltage is within a range between the first voltage and the decomposition initiation voltage of the sulfur oxides, the first voltage being excluded from the range, and the decomposition initiation voltage being included in the range;
obtaining the a second current by the electronic control unit, the second current being the output current detected by the current detector at a time when the applied voltage is a particular voltage that is equal to or higher than a voltage at which the output current is the limiting current of oxygen, and the second current being the output current that exclude a current resulted from the reoxidation reaction of sulfur that has been adsorbed to the first electrode in the first electrode by the boosting sweep; and
calculating, by the electronic control unit, a difference between the obtained second current and the obtained first current and using the difference as the parameter.
